(19) United States
(12) Patent Application Publication
(10) Pub. No.: US 2009/0203901 A1
(43) Pub. Date: Aug. 13, 2009

(54) Process for preparing pyrazole functionalized benzodiazepinones

(75) Inventors: Peter J. Harrington, Louisville, CO (US); Valerie Grace Paulsen, Thornton, CO (US); Charles E. Tucker, Superior, CO (US)

Correspondence Address: Grant D. Green;Roche Palo Alto LLC, Patent Law Department, M/S A2-250, 3431 Hillview Avenue, Palo Alto, CA, 94304, US

(73) Assignee: Roche Colorado Corporation

(21) Appl. No.: 12/378,363

(22) Filed: Feb. 12, 2009

Related U.S. Application Data
(60) Provisional application No. 61/027,893, filed on Feb. 12, 2008.
(60) Provisional application No. 61/033,051, filed on Mar. 3, 2008.

(51) Int. Cl. C07D 487/04; C07C 211/45; C07C 233/33; C07D 243/24

(52) U.S. Cl. 540/495; 564/442; 564/195; 540/504

(57) ABSTRACT

The present invention provides innovative strategies for synthesizing pyrazole ring-functionalized benzodiazepinones. Alternative intermediates and high conversion strategies for forming alpha-aminobenzophenone intermediates involve a combination of aromatic acylation, displacement of electronegative leaving groups with amine, and then N-displacement strategies to produce the desired alpha-aminobenzophenone with primary amine functionality. Reaction strategies are then provided for converting alpha-aminobenzophenones to alpha-aminoamidobenzophenone intermediates with high yield and convenient reaction strategies. These alpha-aminoamidobenzophenone intermediates are then converted into benzodiazepinones. These benzodiazepinones are then converted to pyrazole ring functionalized benzodiazepinones through a series of innovative intermediates and/or reaction strategies.

### CROSS-REFERENCE TO RELATED APPLICATIONS

This application is entitled to the benefit of U.S. provisional patent application Ser. No. 61/027,893 filed on Feb. 12, 2008 and U.S. provisional patent application Ser. No. 61/033,051 filed on Mar. 3, 2008, the disclosures of which are incorporated herein by reference.

### FIELD OF THE INVENTION

The present invention relates to pyrazole functionalized benzodiazepinones, particularly those that inhibit angiogenesis, kinase, and/or mitosis activity. The present invention also relates to reaction strategies and intermediates, including functionalized benzophenones, useful for making the pyrazole functionalized benzodiazepinones.

### BACKGROUND OF THE INVENTION

Angiogenesis is the generation of new blood vessels in a tissue or organ. Under normal physiological conditions, humans and animals undergo angiogenesis only in very specific, restricted situations. For example, angiogenesis is normally observed in wound healing, fetal and embryonal development, and formation of the corpus luteum, endometrium and placenta.

Capillary blood vessels are composed of endothelial cells and pericytes, surrounded by a basement membrane. Angiogenesis begins with the erosion of the basement membrane by enzymes released from endothelial cells and leukocytes. Endothelial cells, lining the lumen of blood vessels, then protrude through the basement membrane. Angiogenic stimulants induce the endothelial cells to migrate through the eroded basement membrane. The migrating cells form a “sprout” off the parent blood vessel where the endothelial cells undergo mitosis and proliferate. The endothelial sprouts merge with each other to form capillary loops, creating a new blood vessel.

Uncontrolled angiogenesis is a hallmark of cancer. In 1971, Dr. Judah Folkman proposed that tumor growth is dependent upon angiogenesis. See, e.g., Folkman, New England Journal of Medicine, 285:1182-86 (1971). According to Dr. Folkman, a tumor can only grow to a certain size without the growth of additional blood vessels to nourish the tumor. In its simplest terms, this proposition states: that “once tumor ‘take’ has occurred, every increase in tumor cell population must be preceded by an increase in new capillaries converging on the tumor.” Tumor ‘take’ is currently understood to indicate a prevascular phase of tumor growth in which a population of tumor cells occupying a few cubic millimeters volume, and not exceeding a few million cells, can survive on existing host microvessels. Expansion of tumor volume beyond this phase requires the induction of new capillary blood vessels. For example, pulmonary micro-metastasis in the early prevascular phase in mice would be undetectable except by high power microscopy on histological sections.

As early as 1945, Algire, et al., J. Nat. Cancer Inst., 6:73-85 (1945), showed that the growth rate of tumors implanted in subcutaneous transparent chambers in mice is slow and linear before neovascularization, and rapid and nearly exponential after neovascularization. In 1966, Dr. Folkman reported that tumors grown in isolated perfused organs where blood vessels do not proliferate are limited to 1-2 mm³ but expand rapidly to >1000 times this volume when they are transplanted in mice and become neovascularized. See, e.g, Folkman, et al, Anals of Surgery, 164:491-502 (1966).

Tumor growth in avascular cornea proceeds slowly and at a linear rate, but switches to exponential growth after neovascularization. See, e.g., Gimbrone, Jr., et al., J. Nat. Cancer Inst., 52:421-27 (1974)). Tumors suspended in the aqueous fluid of the anterior chamber of the rabbit eye remain viable, avascular, and limited in size to <1 mm³. Once they are implanted on the iris vascular bed, they become neovascularized and grow rapidly, reaching 16,000 times their original volume within two weeks. See, e.g., Gimbrone, Jr. et al., J. Exp. Med., 136:261-76.

When tumors are implanted on the chick embryo chorioallantoic membrane, they grow slowly during an avascular phase of >72 hours, but do not exceed a mean diameter of 0.93+0.29 mm. Rapid tumor expansion occurs within 24 hours after the onset of neovascularization, and by day 7 the vascularized tumors reach a mean diameter of 8.0+2.5 mm. See, e.g., Knighton, British, J. Cancer, 35:347-56 (1977)).

Vascular casts of metastasis in the rabbit liver reveal heterogeneity in size of the metastasis, but show a relatively uniform cut-off point for the size at which vascularization is present. Tumors are generally avascular up to 1 mm in diameter, but are neovascularized beyond that diameter. See, e.g., Lien, et al., Surgery, 68:334-40 (1970).

In transgenic mice which develop carcinomas in the beta cells of the pancreatic eyelets, pre-vascular hyperplastic eyelets are limited in size to <1 mm. At 6-7 weeks of age, 4-10% of the eyelets become neovascularized, and from these eyelets arrive large vascularized tumors of more than 1,000 times the volume of the pre-vascular eyelets. See, e.g., Folkman, et al., Nature, 339:58-61 (1989).

It has been shown that tumors can be treated by inhibiting angiogenesis rather than inhibiting proliferation of the tumor cells themselves. For example, Kim et al., Nature, 362:841044 (1993), show that a specific antibody against VEGF (vascular endothelial growth factor) reduces micro-vessel density and causes “significant or dramatic” inhibition of growth of three human tumors which rely on VEGF as their sole mediator of angiogenesis (in nude mice). The antibody does not inhibit growth of the tumor cells in vitro. Further, Hori, et al., Cancer, Resp. 51:6180-84 (1991), shows that anti-bFGF monoclonal antibody causes 70% inhibition of growth of a mouse tumor which is dependent upon secretion of bFGF as its only mediator of angiogenesis. The antibody does not inhibit growth of the tumor cells in vitro. Intraperitoneal injection of bFGF has also been shown to enhance growth of a primary tumor and its metastasis by stimulating growth of capillary endothelial cells in the tumor. The tumor cells themselves lack receptors for bFGF and bFGF is not a mitogen for the tumor cells in vitro. See, e.g., Gross, et al., Proc. Am. Assoc. Cancer Res., 31:79 (1990). A specific angiogenesis inhibitor (AGM-1470) inhibits tumor growth and metastasis in vivo, but is much less active in inhibiting tumor cell proliferation in vitro. It inhibits vascular endothelial cell proliferation half-maximally at 4 logs lower concentration than it inhibits tumor cell proliferation. See, e.g., Ingber, et al., Nature, 48:555-57 (1990).

There is also indirect clinical evidence that tumor growth is angiogenesis dependent. For example, human retinoblastomas that are metastatic to the vitreous develop into avascular spiroids which are restricted to <1 mm³ despite the fact that they are viable and incorporate ³H-Thymidine (when removed from an enucleated eye and analyzed in vitro). Carcinoma of the ovary metastasizes to the peritoneal membrane as tiny avascular white seeds (1-3 mm³). These implants rarely grow larger until one or more of them become neovascularized. Intensity of neovascularization in breast cancer (see, e.g., Weidner, et al., New Eng. J. of Med., 324:1-8 (1991); Weidner, et al., J. Nat. Cancer Inst., 84:1875-87 (1992)) and in prostate cancer (Weidner, et al., Am. J. Pathol., 143 (2):401-09 (1993)) correlates highly with risk of future metastasis.

Metastasis from human cutaneous melanoma is rare prior to neovascularization. The onset of neovascularization leads to increased thickness of the lesion and an increased risk of metastasis. See, e.g., Srivastava, et al., Am. J. Pathol., 133:419-23 (1988)). In bladder cancer, the urinary level of an angiogenic protein, bFGF is a more sensitive indicator of status and extensive disease than is cytology. See, e.g., Nguyen, et al., J. Nat. Cancer, Inst., 85:241-42 (1993).

Angiogenesis has been associated with a number of different types of cancer, including solid tumors and blood-borne tumors. Solid tumors with which angiogenesis has been associated include, but are not limited to, rhabdomyosarcomas, retinoblastoma, Ewing's sarcoma, neuroblastoma, and osteosarcoma. Angiogenesis also has been linked with breast cancer, prostate cancer, lung cancer, and colon cancer. Angiogenesis is also associated with blood-borne tumors, such as leukemias, lymphomas, multiple myelomas, and any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed too that angiogenesis plays a role in the abnormalities in the bone marrow that give rise to leukemia and lymphoma tumors and multiple myeloma diseases.

One of the most frequent angiogenic diseases of childhood is the hemangioma. A hemangioma is a tumor composed of newly-formed blood vessels. In most cases the tumors are benign and regress without intervention. In more severe cases, the tumors progress to large cave and infiltrated forms and create clinical complications. Systemic forms of hemangiomas, hemangiomatoses, which have a high mortality rate. Therapy-resistant hemangiomas exist that cannot be treated with therapies currently in use.

Thus, it is clear that angiogenesis plays a major role in the metastasis of cancer. If this angiogenic activity could be repressed or eliminated, then the tumor, although present, would not grow. In the disease state, prevention of angiogenesis could overt the damage caused by the invasion of the new micro vascular system. Therapies directed at control of angiogenic processes could lead to the abrogation or mitigation of these diseases.

Several classes of compounds that inhibit angiogenesis are being investigated as therapeutic agents. These are, for example, thalidomide and thalidomide analogs (U.S. Pat. Nos. 6,235,756 (The Children's Medical Center Corporation); 6,420,414 (The Children's Medical Center Corporation); 6,476,052 (Celgene Corporation)); quinolinones (U.S. Pat. No. 6,774,237 (Chiron Corporation)); serine proteases and kallikreins (U.S. Pat. No. 6,544,947 (EntreMed. Inc.)); VEGF analogs and antagonists (U.S. Pat. Nos. 6,783,953 (Janssen Pharmaceutica N.V.); 6,777,534 (Children's Medical Center Corporation)); peptides and proteins that bind Angiostatin™ or Endostatin™ (U.S. Pat. No. 6,201,104 (EntreMed. Inc.)); cathepsin V-like polypeptides (U.S. Pat. No. 6,783,969 (Nuvelo Inc.)); other antiangiogenic peptides (U.S. Pat. No. 6,774,211 (Abbott Laboratories); 4-anilino-quinazolines (WO 2002/092578, WO 2002/092577, WO 2002/016352, WO 2001/032651, WO 2000/047212 (Astrazeneca)); phthalazines (WO 2004/033042 (Novartis), WO 2003/022282 (Novartis), WO 2002/012227 (Astrazeneca), WO 2001/010859 (Bayer), WO 98/35958 (Novartis)); isothiazoles (WO 99/62890 (Pfizer)); and indolinones (WO 2001/037820, WO 2000/008202, WO 98/50356, WO 96/40116 (Sugen Inc.)).

Several review articles report the use of angiogenesis inhibitors as therapeutic agents. These articles include Mazitschek et al. Current Opinion in Chemical Biology, 8(4): 432-441 (2004); Underiner, et al., Current Medicinal Chemistry, 11(6): 731-745 (2004); Manley, et al., Biochimica et Biophysica Acta, 1697 (1-2): 17-27 (2004); Alessi, et al., Biochimica et Biophysica Acta, 1654(1): 39-49 (2004); Tortora, et al., Current Pharmaceutical Design, 10(1): 11-26 (2004).

Uncontrolled cell proliferation is another hallmark of cancer. Cancerous tumor cells typically have some form of damage to the genes that directly or indirectly regulate the cell-division cycle.

Cyclin-dependent kinases (CDKs) are enzymes which are critical to cell cycle control. See, e.g., Coleman et al., Annual Reports in Medicinal Chemistry, 32: 171-179 (1997). These enzymes regulate the transitions between the different phases of the cell cycle, such as the progression from the G1 phase to the S phase (the period of active DNA synthesis), or the progression from the G2 phase to the M phase, in which active mitosis and cell-division occurs. See, e.g., the articles on this subject appearing in Science, 274: 1643-1677 (6 Dec. 1996).

CDKs are composed of a catalytic CDK subunit and a regulatory cyclin subunit. The cyclin subunit is the key regulator of CDK activity, with each CDK interacting with a specific subset of cyclins: e.g. cyclin A (CDK1, CDK 2). The different kinase/cyclin pairs regulate progression through specific stages of the cell cycle. See, e.g., Coleman, supra.

Aberrations in the cell cycle control system have been implicated in the uncontrolled growth of cancerous cells. See, e.g., Kamb, Trends in Genetics, 11: 136-140 (1995); and Coleman, supra. In addition, changes in the expression of or in the genes encoding CDK's or their regulators have been observed in a number of tumors. See, e.g., Webster, Exp. Opin. Invest. Drugs, 7: 865-887 (1998), and references cited therein. Thus, there is an extensive body of literature validating the use of compounds inhibiting CDKs as anti-proliferative therapeutic agents. See, e.g. U.S. Pat. No. 5,621,082; EP 0 666 270 A2; WO 97/16447; and the references cited in Coleman, supra, in particular reference no. 10. Thus, it is desirable to identify chemical inhibitors of CDK kinase activity.

It is particularly desirable to identify small molecule compounds that may be readily synthesized and are effective in inhibiting one or more CDKs or CDK/cyclin complexes, for treating one or more types of tumors.

Several classes of compounds that inhibit cyclin-dependent kinases have been and are being investigated as therapeutic agents. These are, for example, analogs of Flavopiridol (U.S. Pat. No. 5,733,920 (Mitotix); WO 98/1344 (Bristol-Myers Squibb); WO 97/42949 (Bristol-Meyers Squibb)); purine derivatives (WO 98/05335 (CV Therapeutics); WO 97/20842 (CNRS)); acridones and benzothiadiazines (WO 98/49146 A2 (US Dept. of Health and Human Services)); and antisense (U.S. Pat. No. 5,821,234 (Stanford University)). Furthermore, certain N,N-substituted dihydropyrazolobenzodiazepines have been disclosed in an article discussing CNS-acting compounds. See, M. A. Berghot, Arch. Pharm. 325:285-289 (1992).

There also continues to be a need for easily synthesized, small molecule compounds for the treatment of one or more types of tumors, in particular through regulation of angiogenesis and/or CDKs. Indeed, the recent U.S. Patent Pub. No. 2006/0079511 describes a particularly interesting class of pyrazole ring-functionalized benzodiazepinone compounds useful as mitosis and angiogenesis inhibitors and their uses in pharmaceutical compositions. These compounds are synthesized through a number of reactions steps involving a number of intermediates. A preferred embodiment of these inhibitors has the formula

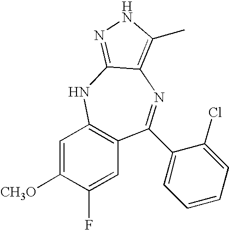

There is a need to improve upon the synthesis strategies used to manufacture these compounds through improved reaction conditions, synthesis strategies that use more widely accessible raw materials, synthesis strategies that improve yields, and the like.

### SUMMARY OF THE INVENTION

The present invention provides innovative strategies for synthesizing pyrazole ring-functionalized benzodiazepinones. Alternative intermediates and high conversion strategies for forming alpha-aminobenzophenone intermediates involve a combination of aromatic acylation, displacement of electronegative leaving groups with amine, and then N-displacement strategies to produce the desired alpha-aminobenzophenone with primary amine functionality. Reaction strategies are then provided for converting alpha-aminobenzophenones to alpha-aminoamidobenzophenone intermediates with high yield and convenient reaction strategies. These alpha-aminoamidobenzophenone intermediates are then converted into benzodiazepinones. These benzodiazepinones are then converted to pyrazole ring functionalized benzodiazepinones through a series of innovative intermediates and/or reaction strategies.

In one aspect, the application provides a method of making a compound of Formula I

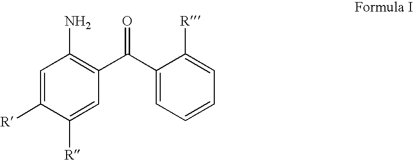

comprising the steps of:

(a) reacting a compound of formula II

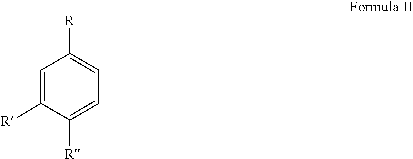

with a compound of Formula III

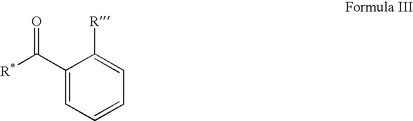

to form a benzophenone of Formula IV

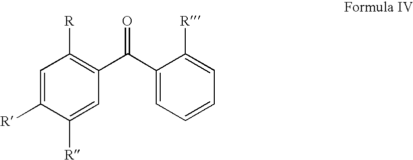

(b) reacting the benzophenone of Formula IV with a benzyl or allyl amine to form a compound of formula V; and

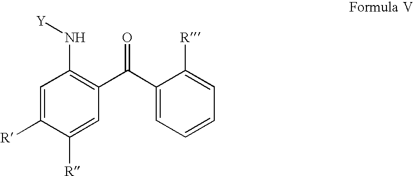

(c) reacting the compound of Formula V with acid to cause N-displacement of Y to form the compound of Formula I;

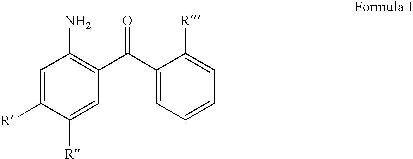

wherein:
Y is benzyl or allyl;
R is halogen, tosylate, or mesylate;
R* is halogen;
R″ is alkyl, alkoxy, halogen, COOH, COAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

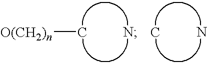

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In another aspect, the application provides a method of forming a compound of Formula I comprising the step of

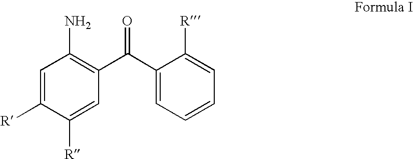

reacting a compound of Formula V

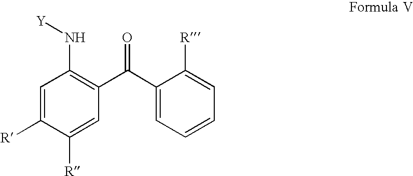

with acid under conditions sufficient to cause hydrogenolysis to form the compound of Formula I;
wherein:
Y is benzyl or allyl;
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

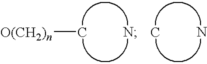

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In one variation of the above method, the solvent is a mixture of methanol and toluene.

In one variation of the above method, the acid is hydrobromic acid.

In one variation of the above method, the conditions sufficient to cause hydrogenolysis comprise the addition of a palladium catalyst.

In one variation of the above method, the palladium catalyst is Pd(OH)₂ on carbon.

In one variation of the above method, the Pd(OH)₂ on carbon is activated with H₂.

In one variation of the above method, the solvent is a mixture of methanol and toluene.

In another variation of the above method, the acid is sulfuric acid, and preferably 98% sulfuric acid.

In one variation of the above method, the conditions sufficient to cause hydrogenolysis comprise a temperature between 80° and 120° C.

In one variation of the above method, the temperature between 80° and 120° C. is a temperature greater than 90° C.

In one variation of the above method, the temperature greater than 90° C. is a temperature greater than 95° C.

In one variation of the above method, the temperature greater than 95° C. is a temperature of 100° C.

In another aspect, the application provides a compound of Formula VI

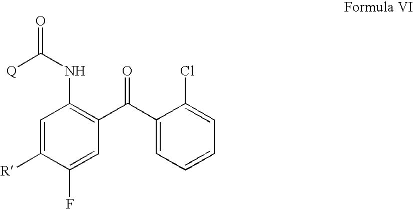

wherein:
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

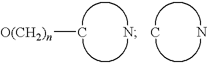

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
Q is (Q′)₂ N(Q″)ᵣ-;
Q′ is lower alkyl, lower alkenyl, lower alkoxy, aryl, aryl alkyl, heteroaryl, heterocyclyl, or cycloalkyl, optionally substituted with one or more substituents selected from the group consisting of halogen, alkyl, OH, and alkoxy;
Q″ is a lower alkylenyl linking group; and
r is 1, 2, or 3.

In one variation of the above compound, R′ is —OMe.

In one variation of the above compound, Q″ is —CH₂— and r is 1.

In one variation of the above compound, wherein Q′ is allyl.

The application further provides any of the above methods, further comprising the step of reacting a compound of Formula I

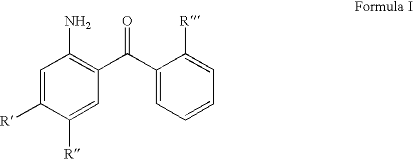

with Q-C(O)O-Q′″ under mild reaction conditions in the presence of a base to form a compound of Formula VI;

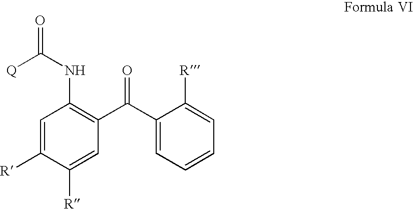

wherein:
Q is (Q′)₂ N(Q″)ᵣ-;
Q′ is lower alkyl, lower alkenyl, lower alkoxy, aryl, aryl alkyl, heteroaryl, heterocyclyl, or cycloalkyl, optionally substituted with one or more substituents selected from the group consisting of halogen, alkyl, hydroxyl, and alkoxy;
Q″ is a lower alkylenyl linking group;
r is 1, 2, or 3;
Q′″ is lower alkyl;
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

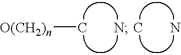

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In one variation of the above method, Q″ is —CH₂— and r is 1.

In one variation of the above method, Q′ is allyl.

In one variation of the above method, Q′″ is ethyl.

In one aspect, the application provides a method of making a compound of Formula VI,

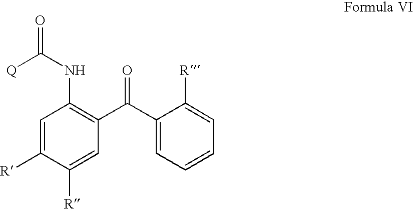

comprising the step of reacting a compound of Formula I

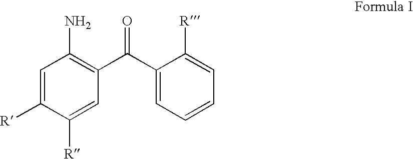

with Q-C(O)O-Q′″ under mild reaction conditions in the presence of a base;
wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

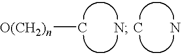

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring;
R′″ is hydrogen, halogen, CN, NO₂, alkyl, or alkoxy;
Q is (Q′)₂ N(Q″)ᵣ-;
each Q′ is independently H, lower alkyl, lower alkenyl, lower alkoxy, aryl, aryl alkyl, heteroaryl, heterocyclyl, or cycloalkyl, optionally substituted with one or more substituents selected from the group consisting of halogen, alkyl, hydroxyl, and alkoxy;
Q″ is a lower alkylenyl linking group;
r is 1, 2, or 3; and
Q′″ is lower alkyl.

In one variation of the above method, the mild reaction conditions include a temperature of between 200 and 30° C.

In one variation of the above method, Q″ is —CH₂— and r is 1.

In one variation of the above method, Q′ is allyl.

In one variation of the above method, Q′″ is ethyl.

In one variation of the above method, the base is a metal alkoxide.

In one variation of the above method, the metal alkoxide is potassium tert-butoxide.

The application further provides any of the above methods, further comprising the step of deallylation-cyclization of the compound of Formula VI

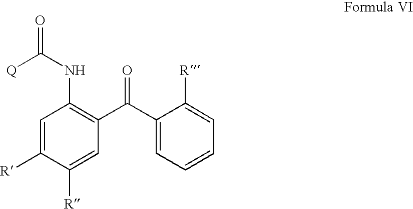

to form a compound of Formula VII

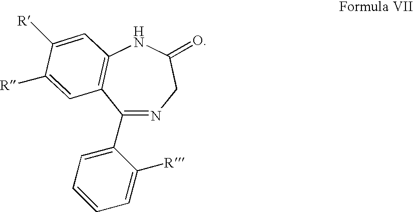

In one aspect, the application provides a method of making a compound of Formula VII,

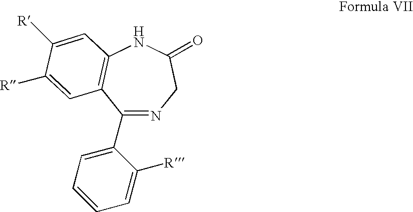

comprising the step of deallylation-cyclization of a compound of Formula VI

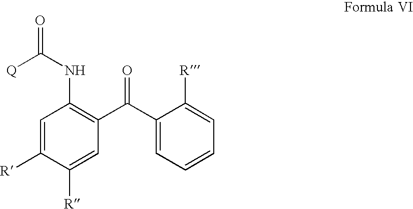

wherein:
Q is (Q′)₂ N(Q″)ᵣ-;

### Q″ is —CH₂—;

Q′ is allyl;
r is 1;
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

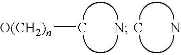

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.
In one aspect, the application provides a method of making a pyrazolo[3,4-b][1,4]benzodiazepine comprising the step of converting a compound of Formula VII

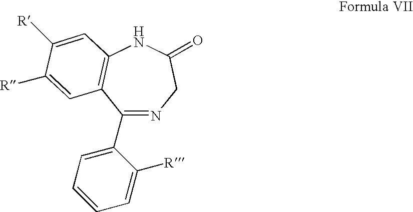

to an imidate of Formula VIIa;

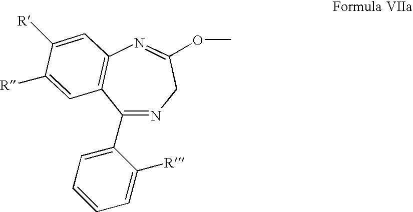

wherein
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

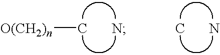

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

The application further provides the above method, further comprising the step of reacting the imidate of Formula VIIa with dimethylacetamide dimethylacetal to form an enamine of Formula VIIb

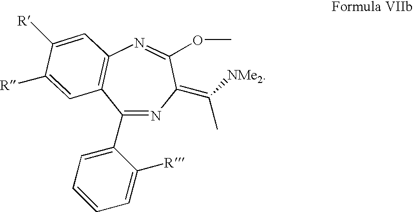

The application further provides the above method, further comprising the step of reacting the enamine of Formula VIb with semicarbazide hydrochloride to form the semicarbazone of Formula VIIc

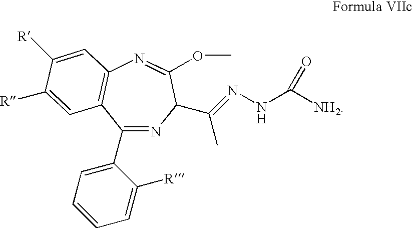

The application further provides the above method, further comprising the step of converting the semicarbazone of Formula VIIc to a carbamate of Formula VIId by acid-catalyzed cyclization

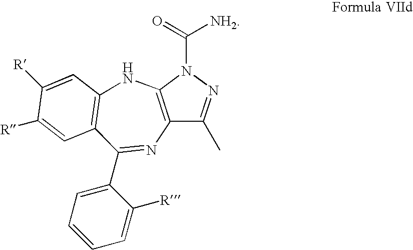

The application further provides the above method, further comprising the step of converting the carbamate of formula VIId to the pyrazolo[3,4-b][1,4]benzodiazepine of Formula VIII by acyl transfer to an amine

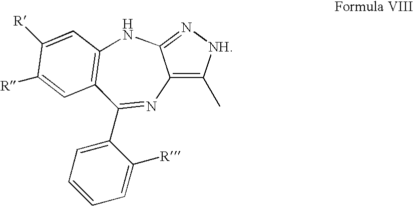

In one aspect, the application provides a method of making a semicarbazone of Formula VIIc comprising the step of reacting an enamine of Formula VIIb

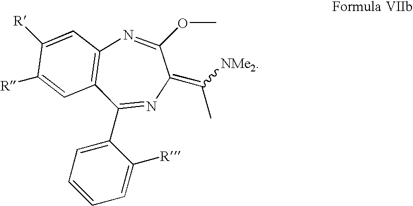

with semicarbazide hydrochloride to form the semicarbazone of Formula VIIc;

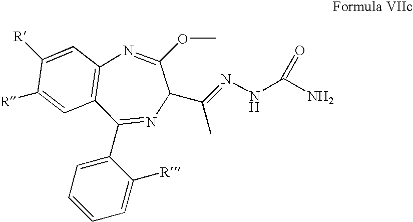

wherein
R″ is alkyl, alkoxy, halogen, COOH, COAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

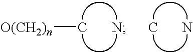

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In one variation of the above method, R′ is methoxy; R″ is F; and R′″ is Cl.

In one aspect, the application provides a method of making a carbamate of Formula VIId

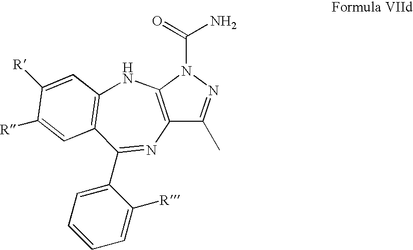

comprising the step of converting a semicarbazone of Formula VIIc

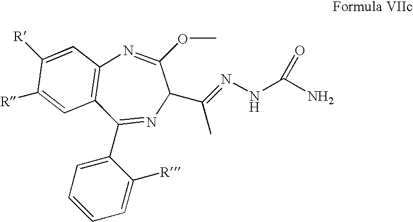

to the carbamate of Formula VIId by acid-catalyzed cyclization;
wherein
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

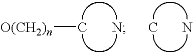

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In one variation of the above method, R′ is methoxy; R″ is F; and R′″ is Cl.

In one variation of the above method, the acid-catalyzed cyclization is HCl-catalyzed cyclization.

In one aspect, the application provides a method of making a pyrazolo[3,4-b][1,4]benzodiazepine of Formula VIII

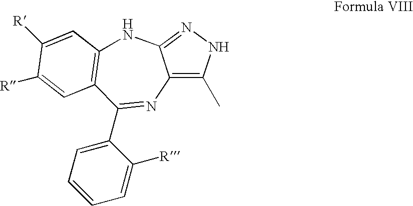

comprising the step of converting a carbamate of formula VIId

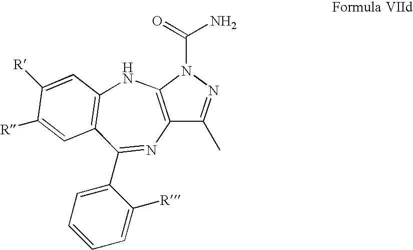

to the pyrazolo[3,4-b][1,4]benzodiazepine of Formula VIII by acyl transfer to an amine.

In one variation of the above method, R′ is methoxy; R″ is F; and R′″ is Cl.

The application further provides the above method, further comprising the step of converting the enamine of Formula VIIb to a ketone of Formula VIIe

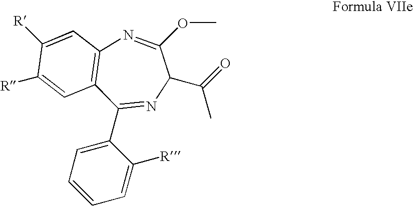

by hydrolysis in methanol.

The application further provides the above method, further comprising the step of reacting the ketone of Formula VIIe with semicarbazide hydrochloride to form a semicarbazone of Formula VIIc

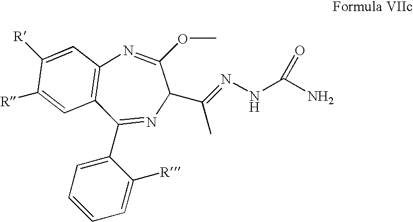

In one aspect, the application provides a compound of the formula VIIe

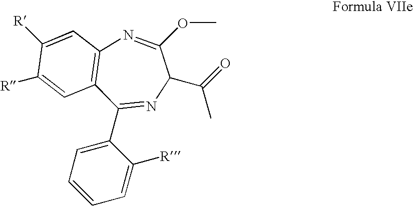

wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

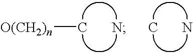

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.
In one variation of the above method,
R′ is methoxy;

### R″ is F; and

### R′″ is Cl.

In one aspect, the application provides a compound of the formula VIIc

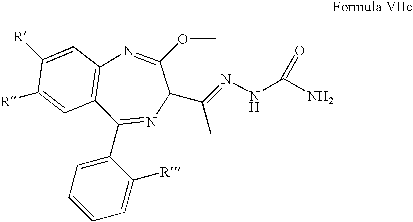

wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

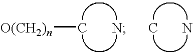

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In one variation of the above method, R′ is methoxy; R″ is F; and R′″ is Cl.

In one aspect, the application provides a compound of the formula VIId

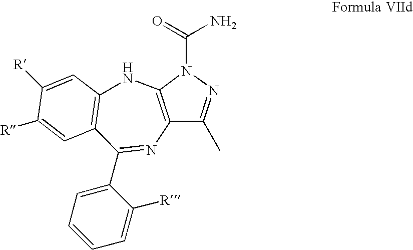

wherein:
R″ is alkyl, alkoxy, halogen, COOH, COAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

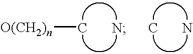

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In one variation of the above method, R′ is methoxy; R″ is F; and R′″ is Cl.

In one aspect, the application provides a method of making a compound of Formula VIIe,

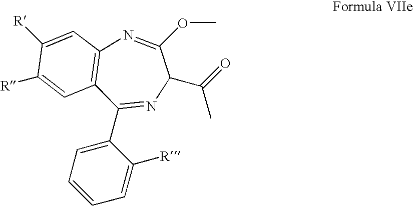

comprising deriving a compound of Formula VIIe from an enamine of Formula VIIb

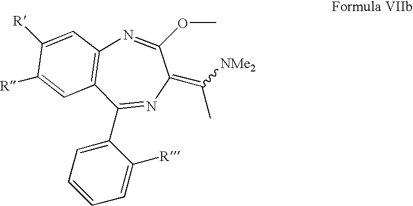

wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

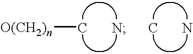

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In one variation of the above method, R′ is methoxy; R″ is F; and R′″ is Cl.

In one aspect, the application provides a method of making a carbamate of Formula VIId,

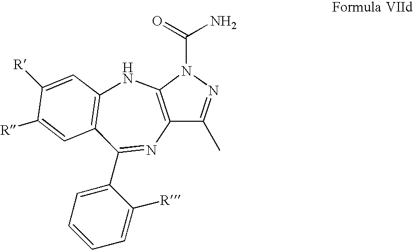

comprising converting a semicarbazone of Formula VIIc

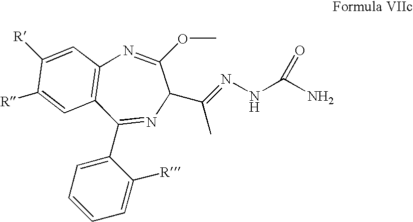

to the carbamate of Formula VIId by acid-catalyzed cyclization wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

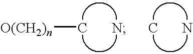

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

The application further provides the above method, further comprising the step of converting the carbamate of formula VIId

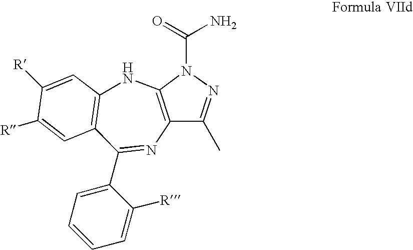

to a pyrazolo[3,4-b][1,4]benzodiazepine of Formula VIII

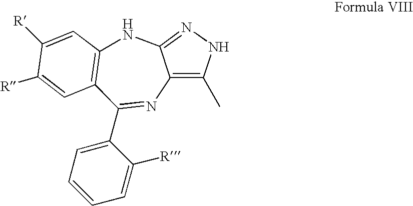

by acyl transfer to an amine.

In one variation of the above method, R′ is methoxy; R″ is F; and R′″ is Cl.

In one aspect, the application provides a method of making a pyrazolo[3,4-b][1,4]benzodiazepine of Formula VIII,

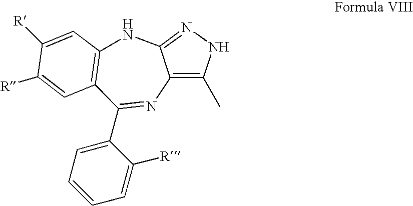

comprising the step of converting a carbamate of formula VIId

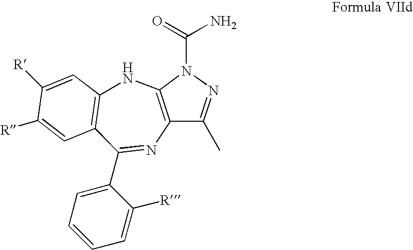

to the pyrazolo[3,4-b][1,4]benzodiazepine of Formula VIII by acyl transfer to an amine wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

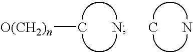

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In one variation of the above method, R′ is methoxy; R″ is F, and R′″ is Cl.

In one variation of the above method, wherein the amine is dimethylamine.

In one variation of the above method, R′ is methoxy; R″ is F, and R′″ is Cl.

In one aspect, the application provides a method of N-debenzylation comprising reacting an N-benzylamine in a solvent with H₂ in the presence of HBr and a palladium catalyst.

In one variation of the above method, the palladium catalyst is Pd(OH)₂ on carbon.

In one variation of the above method, the solvent is a mixture of methanol and toluene.

### BRIEF DESCRIPTION OF THE DRAWINGS

The above mentioned and other advantages of the present invention, and the manner of attaining them, will become more apparent and the invention itself will be better understood by reference to the following description of the embodiments of the invention taken in conjunction with the accompanying drawings, wherein:

FIG. 1 shows alpha-functionalized benzophenones and reaction strategies for preparing these compounds, wherein the compounds are useful in the further synthesis of benzodiazepinones and pyrazole-functionalized benzodiazepinones;

FIG. 2 shows a reaction scheme for converting benzophenones to benzodiazepinones and pyrazole functionalized benzodiazepinones;

FIG. 3 shows a reaction scheme for converting a benzodiazepinone to a pyrazole functionalized benzodiazepinone using innovative reaction strategies and intermediates; and

FIG. 4 shows an exemplary reaction scheme in which an alpha-amino benzophenone is converted to an alpha-aminoamido benzophenone.

### DETAILED DESCRIPTION OF PRESENTLY PREFERRED EMBODIMENTS

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention.

In a first aspect, the present invention relates to novel intermediates and synthesis strategies relating to 2-aminobenzophenones. An aminobenzophenone generally refers to a compound of the formula

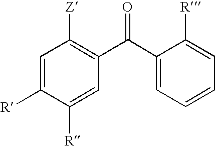

wherein Z′ is a primary, secondary, or tertiary amine functionality, preferably a primary amine functionality; each of R′ through R′″ independently are as follows:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or

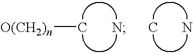

is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

In more preferred embodiments, the 2-aminobenzophenone has the formula

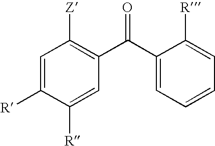

wherein Z′ is an amine group as defined above, R′ is an oxygen containing moiety such as a substituted or nonsubstituted alkoxy group, R″ is an electronegative group such as halide and most preferably is F, and R′″ is an electronegative group such as halide and most preferably is Cl. In a particularly preferred embodiment, the aminobenzophenone has the formula

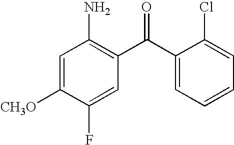

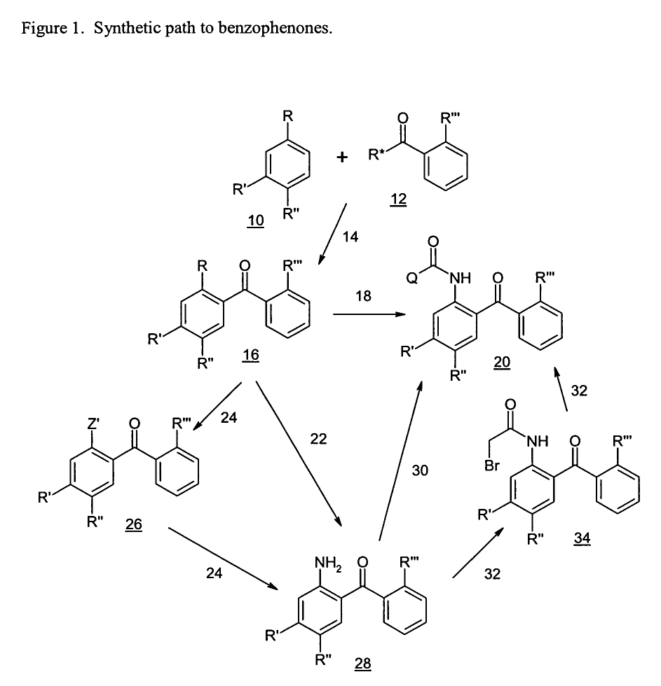

FIG. 1 shows representative reaction schemes of the present invention for preparing 2-aminobenzophenones 28 using principles of the present invention. These representative reaction schemes involve the same first reaction pathway 14. In this first reaction pathway 14, acylation of an aryl compound 10 having an electronegative leaving group R is carried out with an aryl-functional acyl compound 12 to form the leaving group functionalized compound 16.

According to this scheme, the reaction occurs via an electrophilic aromatic substitution reaction to form the desired compound 16 as the reaction product. Electrophilic aromatic substitution, or EAS as the substitution reaction is more widely known, is a reaction in which an atom, usually hydrogen, appended to an aromatic system is replaced by an electrophile. An electrophile is a reagent that is attracted to electrons and that participates in a chemical reaction by accepting an electron pair in order to bond to a nucleophile. Because electrophiles accepts electrons, they are Lewis acids. In preferred modes of practice, acylation is accomplished in this fashion in the presence of a Lewis acid using Friedel-Crafts reaction principles. The reactivity of the electrophile is increased.

Friedel-Crafts acylation has several advantages in the present context of pathway 14. Due to the electron-withdrawing effect of the carbonyl group, the ketone product tends to be less reactive than the original molecule, so multiple acylations tend not to occur.

In compound 10, R′ and R″ are as defined above. R is halogen, tosylate, or mesylate.

In compound 26, Y is benzyl or allyl; In compound 12, R* is halogen and R′″ is as described above.

The electronegative leaving group R may be one or more of halide, tosylate, mesylate, combinations of these, and the like. F is preferred as the leaving group. In the acyl group of compound 12, R′″ is often halide such as Cl or another electronegative moiety compatible with Friedel-Crafts techniques. The reaction product of this first pathway is an alpha-functionalized benzophenone 16 comprising the leaving group R at the alpha position relative to the central keto moiety interconnecting the two aromatic rings.

In preferred modes of practice, the compound 10 has the formula

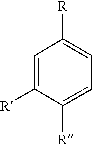

wherein R, R′, and R″ are as defined above. In even more preferred embodiments, compound 10 has the formula

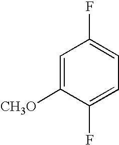

The arrangement and nature of the substituents of this preferred compound advantageously tend to cause the compound to be acylated with high yield at the carbon that is para to the methoxy group. This advantageously positions the F that is meta to the methoxy at the alpha position relative to the resultant, attached keto moiety in the product 16. This in turn allows this more electronegative F to be a preferred leaving group relative to the other F in subsequent reactions described below in which the alpha F is displaced and replaced with amine functionality.

In preferred embodiments, the compound 12 of FIG. 1 has the formula

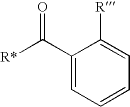

wherein R* and R′″ are as defined above. Each of R* and R′″ independently is halide. In even more preferred embodiments, compound 12 has the formula

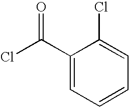

The resultant alpha-functionalized benzophenone preferably has the formula

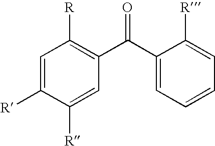

wherein the substituents are as defined above. In more preferred embodiments, the resultant alpha-functionalized benzophenone has the formula

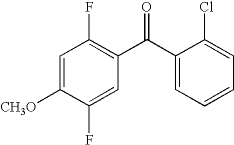

wherein the Cl preferably is alpha to the carbon linked to the keto moiety. More than one such Cl or other halo may be present on this same aromatic ring.

According to one approach to carry out the acylation reaction of pathway 14, the Lewis acid is transferred to a suitable reaction vessel that allows a protective atmosphere, e.g., dry nitrogen, to be maintained. A variety of Lewis acids may be used. This includes AlCl₃, and the like. A variety of solvents may be used. This includes dichloromethane, and the like. Generally, using about 200 ml to 1000 ml of solvent per 5 g to 100 g of the Lewis acid would be suitable.

It is then desirable to chill the contents of the reaction vessel before and/or during the addition of the reactants 10 and 12. Chilling to a temperature in the range of 0 C to 15 C, preferably 0 C to 5 C, would be suitable.

The reactants are then added to the reaction vessel. This may be added singly or in combination. Often, it is desirable to add the acyl functional reactant 12 first and then add the other reactant 10. Each reactant desirably is added to the reaction vessel slowly over a period of time, e.g., 10 seconds to 4 hours, more preferably about 2 minutes to 30 minutes. To minimize the formation of by-products, it is desirable to add the ingredients in close to their stoichiometric amounts. Thus, using a 1:1:1 molar ratio of reactant 10, acyl functional reactant 12, and Lewis acid is preferred. For instance, side products may tend to result in some modes of practice if an excess of aluminum chloride is used.

The vessel contents can be maintained at a chilled temperature over a suitable time period with mixing to allow the reaction to proceed. This desirably occurs at a temperature of from about 0° C. to 15° C. for a time period of from about 3 minutes to 48 hours, more desirably from about 15 minutes to about 5 hours. After this, the contents of the reaction vessel can be further aged at a moderately warmer temperature, e.g., from about 15 C to about 30 C for a period of time in the range of from about 5 minutes to about 48 hours. The reaction product can then be isolated or otherwise carried forward for further reactions.

In representative embodiments, the acylation reaction proceeds to completion with very high yield, e.g., 92% in preferred embodiments such as when the alkoxide functional aromatic compound 10 is 2,5-difluoroanisole and the electrophile is an acid halide such as 2-chlorobenzoyl chloride. The reaction is selective with acylation occurring mainly at the para position relative to the alkoxide. This selectivity results, because the alkoxy group acts as an ortho/para directing group, and substitution at the two ortho positions is inhibited.

In the next stage of synthesis of FIG. 1, the alpha-functionalized product 16 is converted to the desired aminobenzophenone 28. This may occur directly via reaction pathway 22 or via intermediate 26 via reaction pathway 24. Both pathways 22 and 24 are conceptually similar, but pathway 24 provides significant yield advantages even though it involves an extra reaction step. In both pathways 22 and 24, the leaving group R is displaced in favor of an amine moiety substitution. When ammonia is used to carry out the displacement via pathway 22, primary amine-functional compound 28 results directly. However, when a primary or secondary amine, preferably a primary amine, is used to carry out the displacement, intermediate, alpha-aminobenzophenone 26 is obtained in which the substitute amine functionality is either secondary or tertiary. Thus, the moiety Z′ of compound 26 is a secondary or tertiary amine group. When Z′ is a secondary amine group, Z′ is of the general formula NH—Y, wherein Y is alkyl, alkenyl, or aryl alkyl. Examples of amines that can be used in the first part of pathway 24 to form the amine 26 include allylamine, benzylamine, t-butlyamine, and diallylamine, combinations of these, and the like. Of these, allylamine and benzylamine are preferred. Benzylamine is most preferred

The ammonia or amine, as the case may be, desirably displaces a leaving group such as F or other leaving group that preferably is ortho to the keto carbon inasmuch as the leaving group in this ortho position is activated by the nearby keto moiety. Displacement proceeds relatively quickly in the case of amine motivated displacement with high yield when F is the leaving group, the amine reactant is used as at least a portion of the solvent, and the reaction occurs under pressure to minimize formation of by-products.

The fluorine is a preferred leaving group because it is displaced selectively at the ortho position under relatively moderate reaction conditions with high yield. For instance, the F in the ortho position relative to the keto group is displaced selectively with respect to the ortho chlorine of a preferred embodiment because fluorine is more reactive than chlorine under moderate conditions, although some more chlorine displacement would be expected to occur at higher reaction temperatures. Also, the fluorine displacement introduces an amino group, which deactivates the ring system toward further displacement. This also indicates that displacement of the chlorine after the fluorine would require a higher reaction temperature. Other leaving groups such as tosylate (OTs), mesylate (OMs), and triflate (OTf) could be less suitable than F as these others likely could react at least to some degree at sulfur and not at the ring carbon to create undesired by-products.

Although pathway 24 involves an extra reaction step, pathway 24 is more preferred relative to pathway 22 inasmuch as pathway 24 allows higher conversion of intermediate 16 to the alpha-aminobenzophenone 28 in less time. For instance, when fluoride is the leaving group, displacement with 2.0 M ammonia in methanol at 100° C. at 30 to 40 psi might only provide 10% conversion after 20 hours. Without wishing to be bound, it is believed that this relatively slow rate of reaction and limited conversion may be due to imine formation, a context in which the leaving group is more stable and hence harder to displace.

In contrast, higher conversions and/or in shorter periods of time result when amines are used instead of ammonia to displace the leaving group R at the alpha position of compound 16, particularly when the amine is used as at least a portion of the solvent and the displacement is run under pressure to prevent imine formation. For instance, flouride displacement from intermediate 16 with t-butylamine occurs with 70% conversion in 70 hours at 95° C. at 23 psi. Use of other amines is even more favorable. For example, 94% conversion was obtained in only 24 hours using allylamine to displace fluoride under similar conditions. Displacement of fluoride with benzylamine is even better than displacement with allylamine and is preferred. Fluoride displacement with benzylamine in an illustrative embodiment was observed to be complete in just 2 hours at 130° C. Displacement with benzylamine at a much milder 60° C. still accomplishes an 89% conversion with an improved impurity profile after 48 hours. Benzylamine also offers ease of handling and excellent throughput.

Displacement of the leaving group R from compound 16 to yield the intermediate compound 26 using amines may occur in a variety of ways. According to one representative mode of practice using benzyl amine, for example, the benzylamine and compound 16 are combined in a vessel. One suitable vessel set up involves a 2-liter, glass, jacketed Ace pressure reactor with overhead paddle stirrer with a glass shaft and Teflon paddle. The vessel also may be fitted with a Teflon stopper having a pressure gauge, a Teflon stopper with a Teflon valve with septum, and two Teflon stoppers. A large stoichimetric excess of the amine reactant is used so that the amine functions as the reaction solvent. Generally, using from about 50 mmol to about 1000 mmol, preferably 500 mmol to about 750 mmol of the alkoxylated benzophenone reactant per about 50 ml to about 2000 ml, more preferably about 100 ml to about 400 ml would be suitable.

The contents of the reaction vessel may be heated for a suitable period of time under pressure to facilitate the reaction. Generally, heating at a temperature of from about 30° C. up to the boiling temperature of the amine would be suitable. By way of example, the contents of the vessel may be maintained at a temperature in the range of from about 50° C. to 70° C. for about 10 minutes to 72 hours. The reaction product 26 may then be isolated or otherwise further handled or processed.

According to a representative mode of practice for displacement of the leaving group R from compound 16 to yield the intermediate compound 26 using allyllamine, a mixture of the reactant 16 and the allylamine is provided in which the amine is both reactant and solvent. This may be accomplished by using from about 3 to about 20, more preferably from about 8 to about 15 equivalents of the amine reactant per equivalent of the benzophenone reactant 16. The reaction mixture is heated in a pressure vessel at a suitable temperature for a suitable time period to allow the reaction to proceed to the desired degree. Representative reaction temperatures may be in the range of from about 30° C. up to the boiling point of the amine under the pressurized conditions in which the reaction is occurring. Suitable time periods may be in the range of from about 5 minutes to 72 hours, desirably from about 1 hour to about 36 hours. The resultant reaction product may be isolated or otherwise further handled or processed.

Still referring to FIG. 1, the alpha-aminobenzophenone compound 26 formed via pathway 24 is converted to an aminobenzophenone compound 28, for which the amine functionality is primary. In practical effect, the N bound moiety or moieties are removed and replaced with hydrogen(s), yielding the primary amine functionality. The N-displacement reaction can be referenced according to the type of N-bound group to be displaced. For instance, where allyl group(s) are to be displaced, the reaction may be referred to as N-deallylation. Where benzyl groups are to be displaced, the reaction may be referred to as N-debenzylation.

This reaction may be carried out using N-displacement strategies, including conventional techniques as well as innovations of the present invention. For example, to carry out N-deallylation according to one mode of practice, palladium catalyzed allyl group transfer to 1,3-dimethylbarbituric acid converts the allyl functional intermediate 26 to the aminobenzophenone 28 with greater than 90% yield. In more detail, a reaction vessel is charged with the allylamine reactant 26, a suitable acid such as 1.3 dimethylbarbituric acid, catalyst such as palladium supplied in the form of palladium acetate, triphenylphosphine, and solvent. There desirably is a moderate stoichiometric excess of the 1,3-dimethylbarbituric acid to the allylamine embodiments of compound 26 to help ensure that the deallylation goes as far to completion as is practical. In those embodiments that use 1,3-dimethylbarbituric acid, for instance, using from about 1.1 to 5 moles of the acid per mole of the allylamine embodiments of 26 would be suitable. The concentration of the reactants in the solvent may vary over a wide range. In representative embodiments, reaction mixtures may include from about 1 to about 20 moles of allyl amine 26 and 1.1 to about 100 moles of acid per 5 to 200 liters of the solvent. The catalyst is used in conventional catalytic amounts, including amounts in the range from about 0.01 to about 10 moles of catalyst per 50 to 500 moles of allylamine reactant 26.

The ingredients may be heated with mixing for a suitable time period to carry out the N-deallylation reaction. In representative modes of practice, maintaining the mixture at a temperature in the range of 30° C. to about 75° C., more desirably about 35° C., for a time period in the range of from about 1 minute to about 15 hours, more desirably about ten minutes to about 2 hours, would be suitable.

N-debenzylation of the intermediate 26 to provide the primary aminobenzophenone 28 can be accomplished in a variety of ways at favorable yields. As one option, de-benzylation may be carried out under suitable hydrogenolysis conditions such as via hydrogenation or hydrogen transfer from formic acid or ammonium formate. According to such techniques, secondary amines containing an aryl group and a benzyl group are readily reduced by hydrogenation to toluene and the primary aromatic amines.

The technical literature suggests that platinum might be the more desired hydrogenation catalyst when dehydrohalogenation is to be avoided, but the present invention produces the primary aminobenzophenone 28 in high yield, e.g., over about 90% using Pearlman's catalyst (20% palladium hydroxide on carbon) pre-activated under H₂.

In a particularly preferred method, as described herein, the selectivity of palladium catalyzed hydrogenolysis is greatly enhanced in the presence of hydrobromic acid, whereby only trace amounts of des-chloro impurities are observed. The selectivity occurred with a variety of different catalysts, including Engelhard's (Escat series) and Johnson-Matthey's (JM series). The improved selectivity unexpectedly occurred in the presence of hydrobromic acid, but not in the presence of hydrochloric acid, phosphoric acid, or lithium bromide.

De-benzylation may be carried out with some acids under reasonably moderate conditions. For instance, N-debenzylation might not be observed in refluxing trifluoroacetic acid, refluxing 96% formic acid, or in refluxing toluene containing p-toluene sulfonic acid monohydrate. However, N-debenzylation is observed by reacting the compound 26 with concentrated sulfuric acid using from about 0.5 ml to about 100 ml of sulfuric acid per gram of compound 26 at a temperature desirably in the range of from about 25° C. to about 100° C. Addition of a co-solvent such as toluene or mesitylene in an amount up to about 0.1 ml to about 10 ml, more preferably about 0.5 ml per 1 to 5 ml, preferably 2 ml of acid helps to modestly increase yield and also helps provide a less viscous reaction mixture for transfer to a subsequent aqueous quench or other further processing. Additional amounts of co-solvent could be used, except that a reduced reaction rate without commensurate yield improvements may be observed.

The amount of water in the sulfuric acid can impact the reaction rate of de-benzylation in these embodiments. The reaction rate may tend to slow down as the amount of water is increased without a commensurate increase in yield. For example, de-benzylation with 90% by weight sulfuric acid in water is complete in less than about 2 hours at about 100° C., the same reaction with 50% by weight sulfuric acid in water is about ten percent complete in about 4 hours at 100° C.

A preferred mode of practice for carrying out N-debenzylation using sulfuric acid may occur in one or more stages. For example, in a first stage, a suspension of the benzylamine compound 26 in a suitable organic solvent is prepared. Suitable solvents include organic solvents such as toluene, and the like. The suspension desirably includes from about 100 to 1000 mmol of the benzyl amine compound 26 per 10 to about 1800 ml of the reagent. A suitable acid is then added to the reaction vessel. Examples of suitable acids include concentrated sulfuric acid, and the like. The acid can be added slowly over a period of time. Generally, adding from about 2 equivalents to about 20 equivalents, preferably about 10 to 15 equivalents of the acid over a time period of from about 30 seconds to 2 hours would be suitable. Because heat may be generated during addition of the acid, the reaction vessel may be chilled in order to maintain a suitable temperature such as a temperature in the range of from about 20° C. to about 35° C. After completing the acid addition, the mixture may be maintained at such a temperature with stirring for a suitable time period, e.g., from about 1 minute to about 15 hours. In some circumstances, a near-colorless mass may float in the liquid during this stir time.

Next, the suspension may be quickly raised to a higher temperature of from about 85° C. to about 100° C. in order to break up and dissolve the floating mass. The suspension may be maintained at such elevated temperature for a period of time such as from about 1 minute to about 6 hours. After this, the resulting admixture may be cooled and stirred at a temperature in the range of from about 5° C. to about 30° C. for 2 minutes to 24 hours. Optionally, the reaction mixture may be allowed to stand prior to or during transfer to the next stage of the reaction.

In the next stage of the reaction, a two-phase mixture comprising an aqueous phase and an organic phase is prepared in a second vessel. The aqueous phase may include one or more additional water-soluble reagents in addition to water. The organic phase may comprise one or more organic solvents such as toluene, and the like. Desirably, the volume ratio of the aqueous phase to the organic phase may be in the range 1:4 to 4:1, preferably 1:2 to 2:1. The reaction mixture containing the compound 26 is added to the two-phase mixture in the second vessel. This may be done all at once or slowly over a period of time, e.g., from about 20 seconds to about 2 hours. The resultant mixture is then stirred for a period of time, such as from about 5 minutes to about 15 hours, more desirably about 10 minutes to about 3 hours. Then, a base such as aqueous NaOH is added slowly to neutralize the acid and raise the pH to a value such as 13 to 15. More water and/or more organic solvent may need to be added during and/or after the base addition to dissolve precipitated product. During addition of the reaction mixture, the stirring, and the addition of the quenching base, the two phase mixture is maintained at a temperature in the range of 20° C. to about 35° C. to control the exotherm, particularly when base is to be added to quench the acid. If too cool, the reaction product may tend to clump unduly.

The aqueous and organic phases are separated to recover the desired product 28 in the organic phase. The aqueous phase(s) may be washed one or more additional times in order to recover additional amounts of the product. The aqueous phase(s) may need to be moderately heated, e.g., at 30° C. to 40° C., to prevent undue salt precipitation.

The combined organic phases may then be washed one or more times with a suitable aqueous solution such as dilute brine and then concentrated to facilitate recovery of the desired product. The brine content of the washes helps to prevent emulsion formation. Optionally, the product may be recrystallized in a suitable solvent such as ethanol or the like.

Another aspect of the present invention relates to secondary or tertiary amine-functional, 2-amido-benzophenones and methods of making and using these compounds. This aspect relates to the aspects of the invention discussed above in that compounds such as compounds 10, 12, 16, 26, and/or 28 are useful in representative strategies for synthesizing secondary or tertiary amine-functional, 2-amido-benzophenones.

A secondary or tertiary amine-functional, 2-amido-benzophenone refers to a benzophenone comprising a moiety pendant from at least one of the aryl rings at an alpha (ortho) position relative to the ketone moiety linking the first and second aryl moieties, wherein the pendant moiety comprises both an amide group and a pendant secondary or tertiary amine group. Preferably, the amide group links the moiety to the aryl ring.

In representative embodiments, secondary or tertiary amine-functional, 2-amido-benzophenones may be represented by the formula

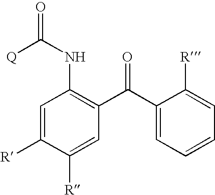

wherein each of R′ through R′″ is as defined above (inasmuch as the secondary or tertiary amine-functional, 2-amido-benzophenones may be derived directly or indirectly from benzophenones 28 and/or 16 that are described above with respect to FIG. 1) and Q is a secondary or tertiary amine functional moiety of the formula

(Q′)₂N(Q″)ᵣ—

wherein,
Q is (Q′)₂ N(Q″)ᵣ-;
each Q′ is independently H, lower alkyl, lower alkenyl, lower alkoxy, aryl, aryl alkyl, heteroaryl, heterocyclyl, or cycloalkyl, optionally substituted with one or more substituents selected from the group consisting of halogen, alkyl, hydroxyl, and alkoxy;
Q″ is a lower alkylenyl linking group; and
r is 1, 2, or 3.

Particularly preferred embodiments of secondary or tertiary amine-functional, 2-amido-benzophenones have the formula

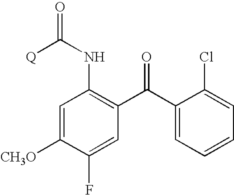

In a particularly preferred embodiment, secondary or tertiary amine-functional, 2-amido-benzophenones have the formula

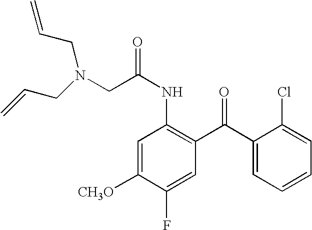

Preferably, the Cl substituent is in an ortho position relative to the keto moiety, but may be meta or para. For this embodiment and throughout this specification, more than one such Cl or other halo may be present on this same aromatic ring. As an alternative to N-allyl moieties, one or more substituted or nonsubstituted aryl, alkyl, or alkoxy groups, e.g., N-benzyl groups, may also be used in such preferred embodiments. The methylene group between the amide and the tertiary amine group may be ethylene or another straight, linear or branched alkylene moiety. The H on the nitrogen of the amide optionally may be replaced with another monovalent moiety. Another alkyl moiety may be used in place of CH₃ in the alkoxy substituent. As alternatives to the F substituent, other suitable electronegative substituents at this position include other halides, and the like.

FIG. 1 shows preferred reaction schemes for preparing a secondary or tertiary amine-functional, 2-amido-benzophenones 20 of the present invention. According to these reaction schemes, the a secondary or tertiary amine-functional, 2-amido-benzophenones 20 may be derived directly from alpha-amino benzophenones 28, directly from alpha-functionalized benzophenones 16, or from benzophonenoes 16 through one or more intermediates such as compounds 28 and/or 26, as the case may be.

According to one reaction pathway 18 shown in FIG. 1, the alpha-functionalized benzophenone 16 is converted directly to a secondary or tertiary amine-functional, 2-amido-benzophenone in a single reaction step. This is accomplished by reacting the benzophenone 16 with a suitable multifunctional reactant comprising a secondary or tertiary amine and an amide moiety. Such a multifunctional reactant can be obtained, for instance, by reacting a haloacetomide with a primary or secondary amine. The amine displaces the halo moiety to yield the aminoacetamide. In a specific example, the reaction of chloroacetamide with diallylamine provides diallylaminoacetamide with an expected yield of about 90%. See: Cheng, C-C.; Yan, S-J. Organic Reactions 1982, 28, 37, whch describes the Friedlander Quinoline Synthesis.

According to an alternative strategy shown in FIG. 1, the secondary or tertiary amine-functional, 2-amido-benzophenones 20 may be formed first by converting benzophenone 16 to the benzophenone 28 via pathway 24 or 22. Then, the benzophenone 28 is converted either directly to the secondary or tertiary amine-functional, 2-amido-benzophenones 20 via pathway 30 or via intermediate alpha-amidated benzophenone 34 via pathway 32. According to the initial portion of this strategy, the benzophenone 16 is converted to the alpha-aminobenzophenone 28 through reaction strategies such as those described above with respect to FIG. 1. For instance, a leaving group such as F at the alpha position of the benzophenone 16 is displaced by reacting with ammonia or amine to form the alpha-amino functional benzophenones 28 or 26, respectively. If the secondary or tertiary alpha-aminobenzophenonses 26 are formed, N-displacement reaction strategies can be used to convert the alpha-amino group to a primary amine moiety.

The resultant alpha-amino benzophenone 28 can be converted to the secondary or tertiary amine-functional, 2-amido-benzophenone 20 via pathway 30 or pathway 32. Pathway 30 provides a direct conversion via a suitable aminolysis reaction in which the benzophenone 28 is reacted with a suitable multifunctional reactant comprising a secondary or tertiary amine and a reactive carbonyl-containing moiety such as an ester moiety, combinations of these, and the like, wherein the reactive carbonyl-containing moiety is one that reacts with amine to form an amide group.

In illustrative embodiments, the multifunctional reactant may have the formula

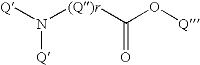

Wherein, Q′ and Q″ are as defined above and Q′″ is lower alkyl, preferably ethyl.

The carbonyl-containing moiety of this reactant forms an amide linkage with the alpha amine of the benzophenone 28.

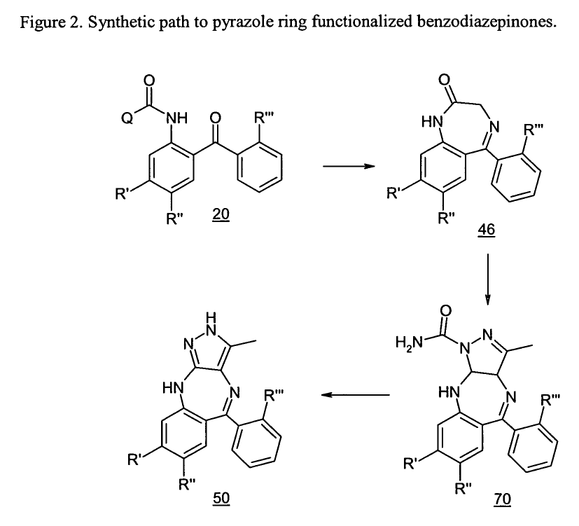

A specific example illustrating how this reaction occurs in typical embodiments is shown in FIG. 2. There, N,N-diallylaminoglycinate 80 is reacted with alpha-aminobenzophenone 82 to form compound 84 directly. The N,N-diallylaminoglycinate 80 is readily obtained by reaction of ethyl acetate with diallylamine. It can be appreciated that other glycinates would be useful in this same reaction scheme and could be easily obtained by reaction of other alkyl acetates with other amines. It can also be appreciated that an ester moiety is not the only carbonyl-containing moiety that will react with amine to form amide.

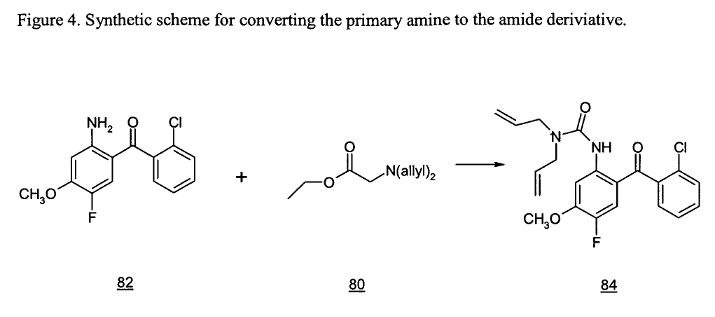

The aminolysis reactions according to pathway 30 such as are shown in FIGS. 1 and 4 may be carried out in a variety of ways. However, the alpha-amino benzophenones 28 tend to be poor nucleophiles. Accordingly, direct amminolyis of a glycinate to form the amide may not be observed merely be mixing the two reactants, even when heating for a period of time, such as at 130° C. for 24 hours. However, the aminolysis reaction proceeds much more effectively in the presence of a polar, nonaqueous, aprotic solvent and a base. Water is desirably excluded from the reaction medium as much as is practical. Examples of polar, organic solvents include tetrahydrofuran (THF); an alcohol such as methanol, ethanol, and/or ispopropyl alcohol (IPA); combinations of these, or the like. Other organic co-solvents such as toluene, or the like may also be used in combination with a polar aprotic solvent. Examples of bases include hexamethyldisilazide, sodium or potassium ethoxide, sodium or potassium tert-butoxide, ______, combinations of these, and the like. In particularly preferred embodiments, the solvent is THF and the base is potassium tert-butoxide.

As examples, a moderate conversion of about 27% has been achieved after 22 hours at 25° C. by reacting 1:1.5 (molar ratio) mixture of the amine 28 with a glycinate in THF using sodium ethoxide in ethanol (1.1 equivalents). High conversion to the amide 20 is observed when sodium hexamethyldisilazide (2.0 equivalents) in THF is added to a stoichiometric mixture of the reactants at −10° C. An 88.7% conversion to amide was obtained when a solution of potassium tert-butoxide (1.25 equivalents) in THF is added to a 1:1.1 (molar ratio) mixture of compound 28 and glycinate at 25° C.

In short, carrying out the aminolysis of alpha-aminobenzophenones in aprotic, polar solvents in the presence of a base at moderate temperatures is rapid and effective. The effectiveness under such mild conditions is unexpected. The technical literature suggests that higher temperatures or microwave radiation would be required. See Varma, R. S.; Naicker, K. P Tetrahedron Lett. 1999, 40, 6177; Perreux, L.; Loupy, A.; Delmotte, M. Tetrahedron, 2003, 59, 2185; Polshettiwar, V.; Kaushik, M. P. Indian J. Chem. 2005, 44B, 773. Mild conditions are a distinct advantage since undesired cyclization to quinolone by-products via mechanisms akin to a Friedlander Synthesis might be observed when larger excesses of base or longer reaction times are used. See Varma et al., Perreux et al., and Polshettiwar et al. articles cited above as well as Wang, J.; Discordia, R. P.; Crispino, G. A.; Li, J.; Grosso, J. A.; Polniaszek, R.; Truc, V. C. Tetrahedron Lett. 2003, 44, 4271; and Cheng, C-C.; Yan, S-J. Organic Reactions 1982, 28, 37. Also, alternative cyclizations to acridone by-products have not been observed using the mild aminolysis conditions of the present invention, which is another by-product cyclization that can occur under more harsh conditions. See Adams, J. H.; Gupta, P.; Khan, M. S.; Lewis, J. R.; Watt, R. A. J. Chem. Soc., Perkin Trans. I 1976, 2089.; Adams, J. H.; Gupta, P.; Khan, M. S.; Lewis, J. R. J. Chem. Soc., Perkin Trans I, 1977, 2173.

To begin a representative mode of practice for carrying out a reaction according to pathway 30, a suspension of the aminobenzophenone compound 28 and the multifunctional reactant is prepared in the dry, organic solvent. Examples of solvents in this context include tetrahydrofuran, and the like. Examples of bases include alkoxide salts such as sodium, potassium, or lithium salts of ethoxide, t-butoxide, and/or the like. Desirably, the multifunctional reactant is present in a slight molar excess, e.g., from about 1.01 to 2.0 moles of the reactant to the aminobenzophenone 28. Enough solvent desirably is used so that the concentration of the aminobenzophenone 28 in the mixture is in the range from about 0.05 to about 5 moles per liter, preferably from about 0.5 to 2 moles per liter. The concentration of the base in the reagent may vary over a wide range, but desirably using about 5 to about 40 weight percent, more preferably 15 to 25 weight percent of base based on the total weight of base plus the reagent would be suitable.

The strong base is then slowly added to the reaction mixture over a period of time such as from about 20 seconds to about 6 hours, more desirably about 3 minutes to about 1 hour. The reaction mixture desirably is maintained at around room temperature or perhaps slightly chilled (e.g., from about 2 C to about 25 C) during addition of the base. Enough base is added so that the reaction mixture includes from about 1 to about 2 equivalents of the base per equivalent of the aminobenzophenone 28. After the addition of base is completed, the reaction mixture may be stirred for a period of time to allow the reaction to proceed to completion as far as is practical. A period of from about 1 minute to about 24 hours, preferably about 10 minutes to about 15 hours would be suitable. The temperature of the reaction mixture may be maintained at about room temperature or just slightly below during this additional period. In an alternative scheme, a strongly basic reagent is prepared first from ingredients including the solvent and base, and then the benzophenone reactant 28 is added to this. Benzyltriethylammonium chloride and additional organic solvent may be added.

According to the other pathway 32 shown in FIG. 1, the alpha-aminobenzophenone 28 is first amidated to convert the alpha-amino group to an amide moiety comprising a leaving group displaceable by reaction with an amine under more moderate conditions. Such leaving groups include halogen, and the like. In preferred embodiments, the leaving group is Br. In a typical reaction, the alpha-aminobenzophenone 28 is reacted with a reactant comprising a carbonyl-containing moiety that forms an amide group upon reaction with amine and the desired leaving group. In many embodiments, this reactant has the formula

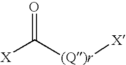

wherein Q″ and r are as defined above, and X and X′ are each independently halogen, combinations of these and the like, preferably Br. A specific embodiment of this reaction occurs when an alpha-aminobenzophenone is reacted with the acyl halide such as BrCH₂C(O)Br to convert the alpha-amino functionality to halo-amido functionality. In the resultant compound 34.

In a representative approach for converting the benzophenone 28 to the intermediate benzophenone 34, a reactant such as an acyl halide is added to a suspension containing the benzophenone 28 in a reagent comprising a base and an organic solvent. Enough base is used so that the amount of base equivalents used in the reagent is substantially the same as the amount of acyl halide equivalents. Enough solvent is used so that the reaction mixture includes from about 10 to about 1000 moles per 10 to 5000 liters of the solvent. In some embodiments, the base is pyridine and the organic solvent is toluene.

A moderate stoichiometric excess of the acyl halide is used relative to the amine to help the reaction go as far to completion as is practical. By way of example, using from about 1.05 to about 3 moles of acyl halide per about one mole of amine would be suitable.

The acyl halide is desirably added slowly over time with stirring while the reaction mixture optionally is slightly chilled. In one mode of practice, adding the acyl halide dropwise over a period of from 15 seconds to 6 hours while the mixture is maintained at 5 C to about 30 C would be suitable. After addition of the acyl halide is complete, the reaction may be stirred for an additional time period in the range from 1 minute to 15 hours, preferably about 5 minutes to two hours, to allow the reaction to proceed. Desirably, the mixture continues to be chilled during this additional time period. The reaction product 34 may then be isolated or otherwise further handled or processed.

Next, as shown by pathway 32 in FIG. 1, the intermediate benzophenone 34 is converted to the desired amino-amido functional benzophenone 20. According to this pathway, the benzophenone 34 is reacted with a primary or secondary amine. This reaction displaces the leaving group pendant from the alpha position, substituting the desired amine group in its place. Examples of suitable amines for converting the compound 34 to the compound 20 include diallylamine, benzylamine, combinations of these, and the like.

In the past, a compound such as benzophenone 34 having a Br leaving group would be reacted with ammonia to displace the Br and replace it with a primary amine functionality. However, such an ammonia reaction might be characterized by only a 60% to 70% yield. In sharp contrast, reaction with an amine occurs easily and may provide a secondary or tertiary amine functional product in near quantitative yields in many embodiments. This amine functionality can then be converted to a primary amine using N-displacement strategies described herein and/or known in the art. Even though the amine/N-displacement approach involves two steps, the overall yield is substantially better than merely reacting with amine. This makes the approach quite desirable when working with expensive materials, such as preferred embodiments of alpha-aminobenzophenones 28.

In a representative mode of practice for converting compound 34 to compound 20 via reaction with an amine, the reaction may be carried out in a solvent. More preferably, excess amine is present to serve as at least a portion of the solvent. By way of example, using from about 5 to 15 equivalents of the amine per equivalent of the haloacylated benzophenone would be suitable. If an additional co-solvent is used, combinations of these, or the like would be suitable. The reaction may occur over a wide range of time periods. Generally, the temperature is above the freezing point and below the reflux temperature of the ingredients. By way of example, the reaction desirably occurs at a temperature in the range of from about 5° C. to about 35° C. for a time period of from about 1 minute to 72 hours, preferably at about room temperature for about 1 to about 24 hours. The product can then be isolated, further processed, or otherwise handled.

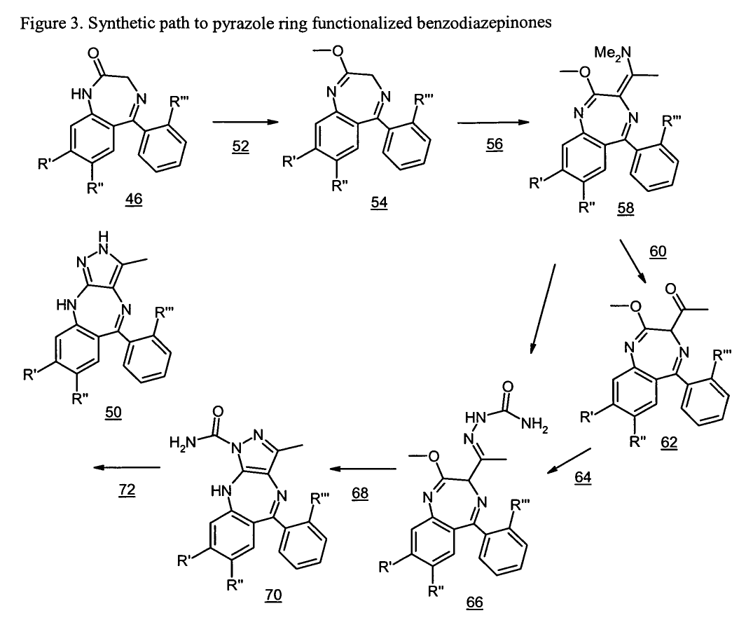

Compounds 20 shown in FIG. 1 have a variety of uses. In one representative mode of use, compounds 20 may be converted to a primary amine compound 42 and then to a benzodiazepinone compound 46 as shown in FIG. 2 via pathways 40 and 44. The aryl diazepinone 46 is then converted into the MAI compound 50 in a series of steps. These steps may be carried out using techniques as described in U.S. Pat. Pub. No. 2006/0079511 (Liu et al.), the entirety of which is incorporated herein by reference for all purposes. Additional innovative procedures of the present invention for converting aryl diazepinone 46 to the MAI inhibitor compound 50 are also described below and shown in FIG. 3.

According to one mode of practice when a preferred embodiment of compound 20 includes one or more N-allyl groups, palladium catalyzed allyl group transfer to 1,3-dimethylbarbituric acid converts compound 20 to the primary amine compound 42. When this transfer is run in refluxing isopropanol in the presence of an acid such as about 20 mol percent acetic, the amine compound is converted in situ to the 1,4-benzodiazepine-2-one compound 46. Alternatively stated, where diallyl amine is used to from intermediate 20, palladium catalyzed allyl group transfer to 1,3-dimethylbarbituric acid in refluxing isopropyl alcohol in the presence of 20 mol % acetic acid yields the aryl diazepinone 46. As a result, the conversion of intermediate 20 to intermediate 46 in this context is believed to proceed through the intermediate 42 shown in FIG. 1.

Compound 46 is a useful intermediate in the synthesis of the MAI inhibitor of formula

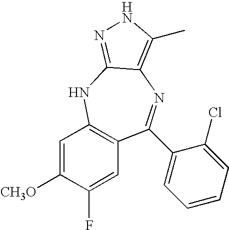

This MAI inhibitor may be viewed as a pyrazole ring-functionalized benzodiazepinone. Thus, in other aspects, the present invention relates to pyrazole ring-functionalized benzodiazepinones and methods of making and using these compounds. These pyrazole ring-functionalized benzodiazepinone-related aspects correspond to the other aspects of the invention described above in that such pyrazole ring-functionalized benzodiazepinones can be synthesized from one or more of the compounds described above including one or more of compounds shown in FIG. 1 such as 16, 28, 20, and/or 46 in FIG. 2.

In the practice of the present invention, a pyrazole ring-functionalized benzodiazepinone generally refers to a benzodiazepinone having a pyrazole ring fused to the seven-membered ring portion of a benzodiazepinone. As used herein, a benzodiazepinone refers to a compound of the formula

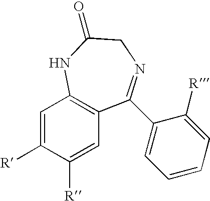

wherein each of R′, R″, and R′″ are as defined above. In preferred embodiments, a benzodiazepinone has the formula

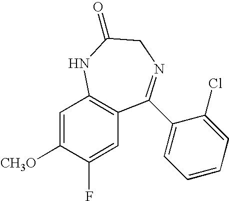

Consequently, a pyrazole ring-functionalized benzodiazepinone of the present invention generally may be represented by the formula

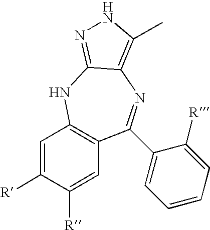

wherein each of the substituents R′, R″, and R′″ are as defined above. In preferred embodiments, a pyrazole ring-functionalized benzodiazepinone has the formula

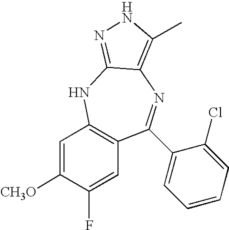

FIG. 3 shows a preferred strategy for synthesizing pyrazole ring-functionalized benzodiazepinones. In a first reaction step 52, the ketone moiety of a benzodiazepinone 46 is converted the keto oxygen to form the substituent OCH₃ and the product 54 of step 52 may be referred to as an imidate. This reaction may be carried out using techniques known in the art, such as are described in US Published Application No. 2006/079511, incorporated herein by reference in its entirety for all purposes. An Example is also included below.

The benzodiazepinone 46 used as a starting material for step 52 may be obtained in a variety of ways. According to representative modes of practice, the starting material 46 may be synthesized from starting materials such as compound 10, 12, 16, 28, 26, and/or 20 (FIG. 1) as described herein. In accordance with preferred embodiments, the product 54 has the formula

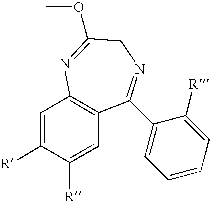

wherein the substituents are as defined above. Preferably, the product 54 has the formula

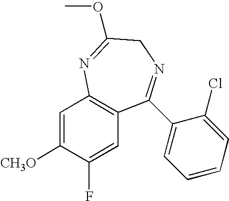

In step 56, the product 54 is converted to enamine 58. This may occur, for example, by the slow addition of an acetamide functional acetal reactant such as dimethylacetamide dimethylacetal (DMA-DMA) of the formula

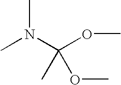

Reaction step 56 desirably occurs by adding the acetamide functional acetal reactant to an admixture including the compound 54 over an extended time period with heating. Desirably, the addition involves adding a substantial stoichiometric excess over several hours, e.g., 24 hours at an elevated temperature such as 130° C. The reaction may occur in a solvent such as DMF. Side products such as diamine and ethyl enamine may form. As noted below, the diamine is basic and has utility in a subsequent synthesis step, described below, if desired. Otherwise, the desired enamine product 58 can be separated from the DMF, by-products, and other impurites in a variety of ways, taking care to minimize hydrolysis or other undue degradation of the enamine 58. On a smaller scale, concentration to dryness may be used, such as by using rotary evaporation techniques at a reduced pressure, e.g., 2 mmHg. At larger scales, hydrolysis of the enamine 58 is relatively slow in toluene at about 25° C. This allows a water/toluene quench at about 25° C. that cleanly separates the enamine 11 as well as the diamine and enamine byproducts from the DMF with minimal enamine hydrolysis.

In preferred embodiments, the enamine reaction product 58 has the formula

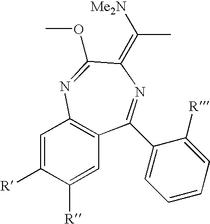

wherein all the substituents are as defined above. In more preferred embodiments, the enamine product 58 has the formula

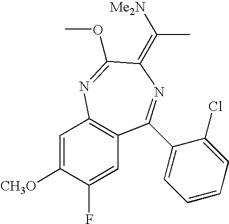

Techniques for carrying out reaction steps 52 and 56 are described in US Published Application No. 2006/079511, incorporated herein by reference in its entirety for all purposes. Examples are also included below.

In step 60, the enamine 58 is hydrolyzed, removing the unsaturation and converting the amine moiety to a ketone 62. Whereas hydrolysis is relatively slow in toluene at 25° C., hydrolysis occurs relatively rapidly in the presence of water in a polar organic solvent such as methanol or the like. Accordingly, switching solvents from the toluene used to quench in step 56 to methanol, followed by a stoichiometric addition of water, facilitates rapid hydrolysis at about 25° C. The resulting ketone product 62 precipitates in the methanol. While one stereoisomer of compound 62 is shown, the desired product may include the other stereoisomer or a mixture of these.

In preferred embodiments, the product 62 preferably has the formula

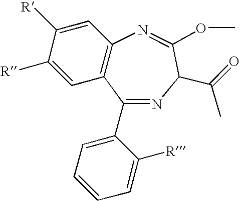

wherein each of the substituents is as defined above. In more preferred embodiments, the ketone product 62 has the formula

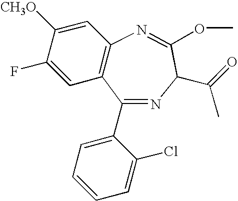

Preferably, the chlorine is at the alpha position relative to the carbon that is linked to the seven-membered ring.

In step 64, the ketone 62 is converted to a semicarbazone of formula 66. As used herein, a semicarbazone refers to a compound comprising a moiety of the formula

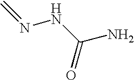

indicating that the semicarbazone moiety is attached to the rest of the molecule by the double bond.

The ketone 62 is converted to semicarbazone 66 in step 64 by reacting with a hydrazine reagent such as semicarbazide hydrochloride. Suitable solvents include polar inorganic solvents such as alcohols, including methanol. Mixtures of methanol and water would also be suitable. A wide range of bases may be used, including sodium bicarbonate, and the like. The semicarbazone 66 may also be generated directly from the enamine 58 by treatment with semicarbazide hydrochloride in methanol at 25° C.

In preferred embodiments, the semicarbazone product 66 has the formula

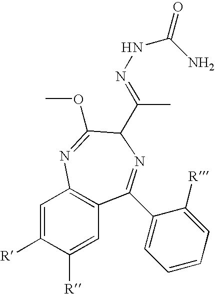

wherein the substituents are as defined above. In even more preferred embodiments, the hydrazide 66 has the formula

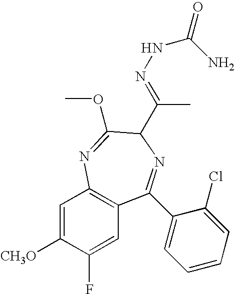

wherein the Cl preferably is at the alpha position relative to the carbon attached to the seven-membered ring. More than one such Cl or other halo may be present on this same aromatic ring.

In step 68, the hydrazide 66 is subjected to a cyclization reaction in which the semicarbazone forms at least a portion of a fused ring structure with respect to the seven-membered ring to form the cyclized product 70. This occurs in the presence of an acid even at about 25° C. For instance, when semicarbazide hydrochloride is used to convert a preferred embodiment of compound 62 to compound 66 in methanol/water, rapid precipitation of a bright orange precipitate of the cyclized product 70 forms in situ at about 25° C. This supports an inference that acid catalyzes the cyclization. Acid also is observed to promote the cyclization of compound 66 to compound 70 in neat methanol.

In preferred embodiments, the cyclized product 70 has the formula

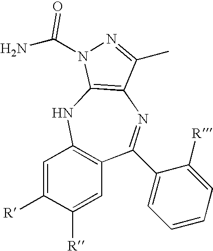

wherein the substituents are as defined above. In more preferred embodiments, the product 70 has the formula

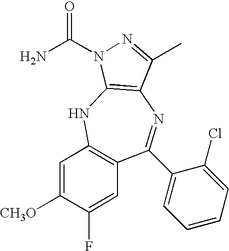

wherein the Cl is desirably alpha to the carbon that is linked to the seven-membered ring. More than one such Cl or other halo may be present on this same aromatic ring.

Next, the cyclized product 70 is converted to the pyrazole ring-functionalized benzodiazepinone 50 in step 72. This is may be accomplished simply by adding an excess of base such as dimethylamine or the like. The compound 70 need not be isolated, so this reaction may occur in the same suspension, e.g., methanol suspension, resulting from the formation of compound 70 from compounds 66 and/or 62.

In preferred embodiments, the resulting compound 50 has the formula

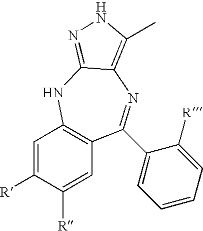

wherein R′, R″, and R′″ are as defined above. In more preferred embodiments, the product 50 has the formula

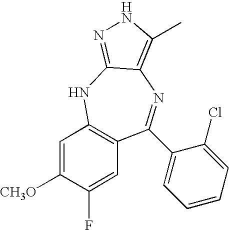

The present invention will now be described with reference to the following illustrative examples.

### EXAMPLES

### Example 1

### 2′-Chloro-2,5-difluoro-4-methoxybenzophenone

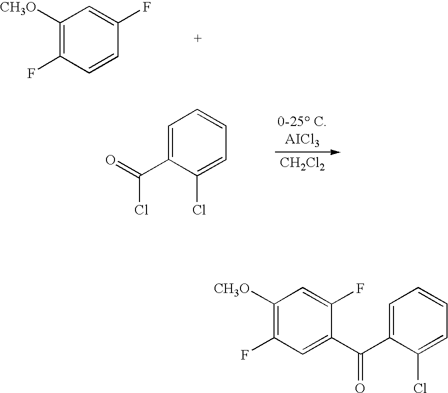

Aluminum chloride (92.54 g, 694 mmol) was transferred to a bottle (in a glove bag under dry N₂), then to the reaction flask. Dichloromethane (600 mL) was added via syringe and the suspension cooled to 0° C. (ice-H₂O bath). 2-Chlorobenzoyl chloride (88.2 mL, 121.45 g, 694 mmol) was added via syringe at 0-5° C. over 13 min. The addition funnel was rinsed with 10 mL dichloromethane. The difluoroanisole (77.8 mL, 100.0 g, 694 mmol) was then added dropwise via syringe at 0-5° C. over 13 min. The resulting yellow solution was stirred at 0-5° C. for 3.5 h (until the bath warmed) then at 20-25° C. for 14 h.

The solution is poured over 600 g ice in a 2 L round bottom flask. Dichloromethane (80 mL) was used to complete the transfer. The layers were separated and the aqueous layer was extracted with 80 mL dichloromethane. The combined organic layers were transferred to a 1 L flask (with paddle stirrer) for distillation under dry N₂. The dichloromethane is then replaced by n-heptane. At a bath temperature of 55-65° C., the head temperature is 35-38° C. and the pot temperature climbs to as high as 52° C. The pot solution is cooled and n-heptane (400 mL) is added. The distillation is continued. At a bath temperature of 100° C., the head temperature climbs from 55 to 75° C. and pot temperature climbs to as high as 83° C. before stopping the distillation (collected 767 mL total distillate). The pot suspension is now two phases, colorless on top and yellow on bottom. The suspension is allowed to cool to 50° C. and stirred at 200 rpm for 1 h. The suspension is allowed to cool to 40° C. and stirred at 200 rpm for 1 h. The suspension is allowed to cool to 30° C. and stirred at 200 rpm for 1 h. The 30° C. suspension is then cooled to 0-5° C. and stirred for 30 min. The precipitate is suction filtered (600 ml coarse sintered glass funnel), washed with 100 mL n-heptane at 0° C., and air dried 17 h at 25° C. to afford 181.23 g (92.4%) of light yellow solid.

Recrystallization from hexanes provides colorless crystals, m.p. 84-85° C.; 500 MHz ¹H NMR (CDCl₃) δ 7.56 (dd, J=6.5 Hz, J=11.5 Hz, 1H), 7.43-7.39 (m, 3H), 7.38-7.34 (m, 1H), 6.65 (dd, J=6.6 Hz, J=11.5 Hz, 1H), 3.94 (s, 3H); 125 MHz ¹³C NMR (CDCl₃) δ 190.2, 159.2 (dd, J=2 Hz, J=253 Hz), 153.9, 148.7 (dd, J=3 Hz, J=244 Hz), 140.0, 131.7, 131.21, 131.19, 130.2, 129.08, 129.07, 127.1, 117.5 (dd, J=3 Hz, J=21 Hz), 101.7 (dd, J=2 Hz, 29 Hz), 56.9; IR (KBr) 3057, 2986, 2941, 1663, 1653, 1620, 1519, 1443, 1420, 1360 cm⁻¹. Elem. Anal. Calcd for C₁₄H₉ClF₂O₂: C, 59.49; H, 3.21; Cl, 12.54; F, 13.44. Found: C, 59.49; H, 3.13.

### Example 2

### 2-(Benzylamino)-2′-chloro-5-fluoro-4-methoxybenzophenone

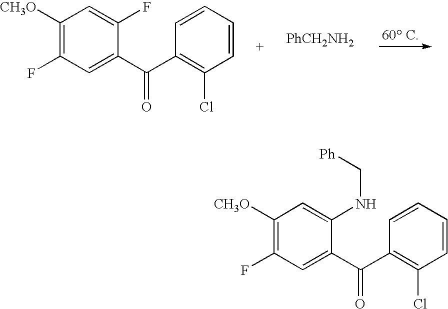

A mixture of 179.58 g (635.3 mmol) of the difluorobenzophenone and 277 mL (272.3 g, 2.54 mol, 4.00 equiv) of benzylamine was heated at 60° C. for 48 h then stirred at 25° C. for 70 h. The suspension was diluted with 800 mL heptane and 800 mL H₂O and stirred for 60 min. The precipitate was suction filtered (600 mL coarse sintered glass funnel), washed with 200 mL H₂O then 200 mL heptane, and air dried 5 h at 25° C. to afford 208.33 g (88.7%) of yellow solid.

Recrystallization from isopropanol provides bright yellow crystals, m.p. 122-124° C.; 500 MHz ¹H NMR (CDCl₃) δ 9.65 (br t, J=5.5 Hz, NH), 7.6-7.27 (m, 9H), 6.86 (d, J=12.5 Hz, 1H), 6.12 (d, J=7.5 Hz, 1H), 4.53 (d, J=5.5 Hz, 2H), 3.77 (s, 3H); 125 MHz ¹³C NMR (CDCl₃) δ 194.9 (d, J=3 Hz), 154.9 (d, J=12.4 Hz), 151.2, 143.1 (d, J=234 Hz), 139.9, 138.3, 130.9, 130.5, 130.2, 129.1, 128.6, 127.7, 127.3, 127.0, 120.5 (d, J=18.6 Hz), 109.4 (d, J=5.3 Hz), 95.4, 56.1, 47.6; IR (KBr) 3301, 3082, 3013, 2939, 1634, 1608, 1566, 1525, 1450, 1257, 1237, 1128 cm⁻¹. Elem. Anal. Calcd for C₂₁H₁₇ClFNO₂: C, 68.20; H, 4.63; Cl, 9.59; F, 5.14; N, 3.79. Found: C, 68.23; H, 4.53; N, 3.75.

### Example 3a

### 2-Amino-2′-chloro-5-fluoro-4-methoxybenzophenone

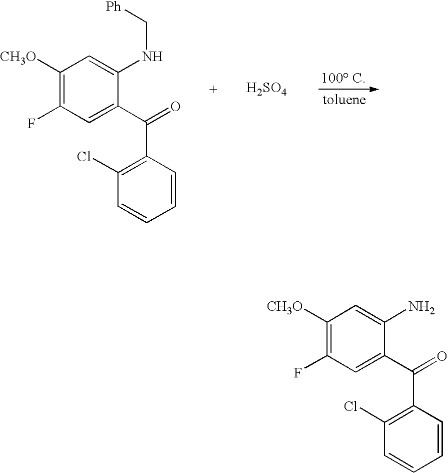

To a suspension of 166.11 g (449 mmol) of the benzylamine and 47.8 mL (41.4 g, 449 mmol) toluene added 330 mL (607 g, 6.19 mol, 13-14 equiv) of concentrated sulfuric acid at 25-35° C. over 10 min (ice-water bath). The suspension is stirred at 25-30° C. for 60 min. The suspension is then quickly heated to 95-100° C. (over 23 min) then maintained at that temperature for 60 min. The syrup is cooled and stirred at 20-25° C. for 2 h then left standing for 33 min during the transfer into water.

The syrup is added via Teflon cannula over 33 min to a mixture of 825 mL H₂O and 500 mL toluene. The temperature is maintained at 25-35° C. during the transfer (ice-water bath). The quench suspension is then stirred at 25-30° C. for 94 min. A solution of 495 g (12.4 mol) of NaOH in 825 mL H₂O is added dropwise at 25-32° C. over 1 h (ice-water bath). 500 mL toluene and 100 mL H₂O are added to dissolve any precipitated product. The layers are separated (aqueous pH is 13-14) and the 30° C. aqueous layer extracted with 100 ml toluene three more times. The extraction mixture is kept at 30° C. to prevent salt precipitation (7.5 M in Na₂SO₄) and plugging of the separatory funnel. The combined organic layers are washed with 100 mL dilute brine H₂0 (16 mL per 100 mL) twice and concentrated in vacuo (rotary evaporator at 30° C. and 25-20 mm Hg then vacuum pump at 25° C. and 1 mm Hg for 3 h) to afford 106.69 g (84.9%) of light yellow solid.

Recrystallization from ethanol provides beige crystals, m.p. 130.5-132.5° C.; 500 MHz ¹H NMR (CDCl₃) δ 7.45 (dd, J=7.0 Hz, J=1.5 Hz, 1H), 7.39 (dt, J=7.5 Hz, J=2.0 Hz, 1H), 7.35 (dt, J=7.5 Hz, J=1.5 Hz, 1H), 7.29 (dd, J=7.5 Hz, J=1.5 Hz, 1H), 6.82 (d, J=12 Hz, 1H), 6.5 (br, 2H, NH), 6.18 (d, J=7.0 Hz, 1H), 3.89 (s, 3H); 125 MHz ¹³C NMR (CDCl₃) δ 195.0 (d, J=2.4 Hz), 154.6 (d, J=12.4 Hz), 150.5, 143.9 (d, J=234 Hz), 139.7, 130.8, 130.6, 130.2, 128.5, 127.0, 119.6 (d, J=18.6 Hz), 109.6 (d, J=4.8 Hz), 99.5, 56.3; IR (KBr) 3487, 3328, 2936, 1624, 1592, 1544, 1513, 1284, 1250, 1117 cm⁻¹. Elem. Anal. Calcd for C₁₄H₁₁ClFNO₂: C, 60.12; H, 3.96; Cl, 12.67; F, 6.79; N, 5.01. Found: C, 59.69; H, 3.91; N, 5.07.

### Example 3b

### 2-Amino-2′-chloro-5-fluoro-4-methoxybenzophenone

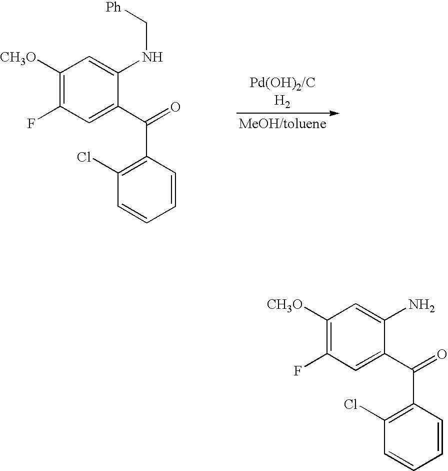

To the benzylamine (20.0 g) and Pd(OH)₂/C (Pearlman's Catalyst (18 wt % Pd/30 wt % water, Degussa)) (1.00 g, 0.05 g/g) in 60.0 mL MeOH/240 mL toluene in a 1 L glass pressure reactor purged with 3×22 psig N₂, was added HBr (1.82 g, 48 wt % in water) and the reaction mixture was then purged with H₂ (3×35 psig), pressurized to 25 psig H2, warmed to 30 C and the pressure increased to 35 psig H₂, and vented every 2 h, for the duration of 8 h, until the conversion to the amine was deemed complete. The mixture was then filtered through celite to give a pale yellow solution (46.8 mg amine/304 g total weight, 94%).

### Example 4

### 2-Bromo-2′-(2-chlorobenzoyl)-4′-fluoro-5′-methoxyacetanilide

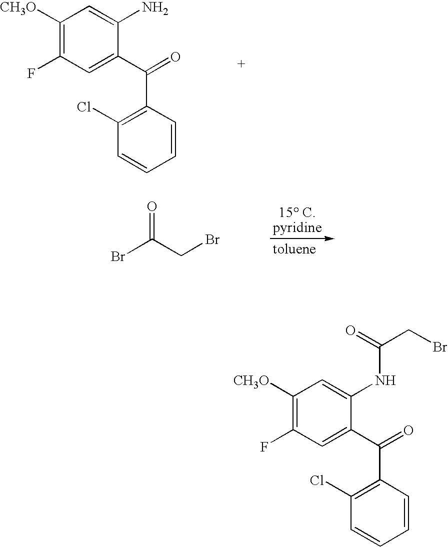

Bromoacetyl bromide (36.1 mL, 83.9 g, 416 mmol, 1.10 equiv) is added dropwise at 13-17° C. (ice H₂O bath) over 24 min to a suspension of 105.69 g (377.9 mmol) of the amine and 33.7 mL (33.0 g, 417 mmol, 1.10 equiv) pyridine in 600 mL toluene. The resulting suspension is stirred at 13-17° C. for 1 h.

The suspension is cooled to 0-5° C. and cold (5° C.) H₂O (600 mL) is added. The suspension stirred at 0-5° C. for 30 min. The precipitate is suction filtered, washed with 150 mL of 5° C. H₂O and 150 mL of 5° C. toluene, and air dried 21.5 h at 25° C. to afford 146.56 g (96.8%) of colorless solid.

Recrystallization from isopropanol and then from toluene provides pale yellow crystals, m.p. 177-178° C.; 500 MHz ¹H NMR (CDCl₃) δ 12.4 (s, 1H, NH), 8.62 (d, J=8.0 Hz, 1H), 7.50-7.44 (m, 2H), 7.41-7.38 (m, 1H), 7.33 (dd, J=8.0 Hz, J=1.5 Hz, 1H), 7.07 (d, J=11.5 Hz, 1H), 4.08 (s, 2H), 4.01 (s, 3H); 125 MHz ¹³C NMR (CDCl₃) δ 197.1 (d, J=1.9 Hz), 166.0, 154.0 (d, J=10.9 Hz), 147.1 (d, J=244 Hz), 139.9 (d, J=2.4 Hz), 138.5, 131.6, 131.0, 130.4, 128.7, 127.2, 120.9 (d, J=19.5 Hz), 114.8 (d, J=4.8 Hz), 105.0, 56.8, 29.8; IR (KBr) 3461, 3116, 1685, 1635, 1624, 1585, 1518, 1344, 1246 cm⁻¹. Elem. Anal. Calcd for C₁₆H₁₂BrClFNO₃: C, 47.97; H, 3.02; Br, 19.94; Cl, 8.85; F, 4.74; N, 3.50. Found: C, 47.93; H, 2.88; N, 3.51.

### Example 5

### 2-(N,N-Diallylamino)-2′-(2-chlorobenzoyl)-4′-fluoro-5′-methoxyacetanilide

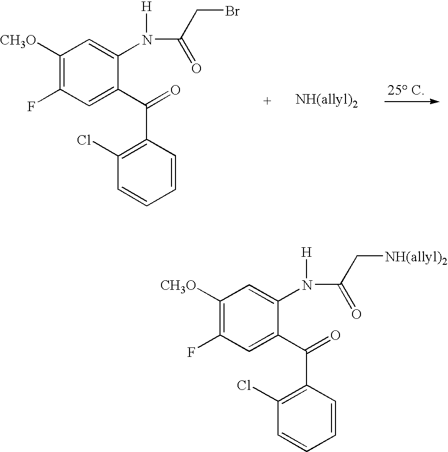

A suspension of 144.90 g (361.7 mmol) of the bromoacetanilide and 356 mL (281 g, 2.89 mol, 8.0 equiv) diallylamine is stirred at 20-25° C. for 15.5 h (cool H₂O bath).

Some excess diallylamine (129.4 g) is distilled at (bath temperature to 32° C.) 20 mm Hg to afford a stirrable syrup. Toluene (625 mL) and a solution of 42.2 g (398 mmol) sodium carbonate in 375 mL H₂O are added simultaneously. After stirring for 60 min, the layers are separated. The organic layer is washed with 200 mL H₂O three times then concentrated in vacuo (rotary evaporator at 30-50° C. and 20 mm Hg, isopropanol trituration, rotary evaporator at 30° C. and 40-15 mm Hg, then vacuum pump at 25° C. and 1 mm Hg for 16.5 h) to afford 146.79 g (97.4%) of light yellow solid.

Recrystallization from isopropanol affords near-colorless crystals, m.p. 90.5-92.5° C.; 500 MHz ¹H NMR (CDCl₃) δ 8.76 (d, J=8.0 Hz, 1H), 7.48-7.42 (m, 2H), 7.39 (dt, J=2.0 Hz, J=7.0 Hz, 1H), 7.34 (dd, J=2.0 Hz, J=7.0 Hz, 1H), 7.03 (d, J=12.5 Hz, 1H), 6.06-5.98 (m, 2H), 5.24 (dm, J=16.5 Hz, 2H), 5.17 (dm, J=10.0 Hz, 2H), 4.00 (s, 3H), 3.29 (s, 2H), 3.27 (d, J=7.0 Hz, 4H); 125 MHz ¹³C NMR (CDCl₃) δ 196.0 (d, J=2.0 Hz), 172.8, 153.6 (d, J=10.9 Hz), 146.6 (d, J=242 Hz), 139.8 (d, J=2.4 Hz), 139.3, 135.2, 131.2, 130.9, 130.3, 128.5, 127.2, 120.5 (d, J=19.5 Hz), 118.9, 114.9 (d, J=4.3 Hz), 105.0, 58.5, 58.2, 56.7; IR (KBr) 3188, 3077, 2978, 2821, 1697, 1638, 1625, 1574, 1520, 1477, 1346, 1296, 1274, 1119 cm⁻¹. Elem. Anal. Calcd for C₂₂H₂₂ClFN₂O₃: C, 63.39; H, 5.32; Cl, 8.50; F, 4.56; N, 6.72. Found: C, 63.53; H, 5.16; N, 6.66.

### Example 6

### 2-(N,N-Diallylamino)-2′-(2-chlorobenzoyl)-4′-fluoro-5′-methoxyacetanilide

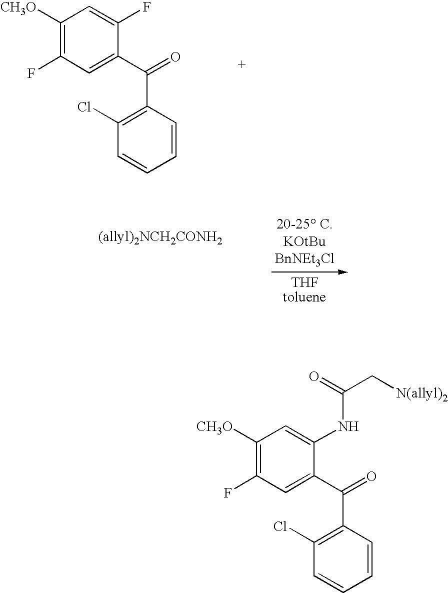

Potassium tert-butoxide in THF (6.0 mL, 5.72 g of 20% wt, 1.144 g KOtBu, 10.2 mmol, 3.0 equiv) was added dropwise at 25° C. to a solution 1.573 g (10.2 mmol, 3.0 equiv) of the amide in 5 mL of dry toluene. The resulting solution was heated to reflux (bath 110° C.) then THF (and tert-butanol) was removed by distillation (collected 6.3 mL). Benzyltriethylammonium chloride (155 mg, 0.680 mmol, 20 mol %) and 5 mL dry toluene were added to the residue. A solution of 0.962 g (3.40 mmol) of the fluorobenzophenone in 5 mL dry toluene was then added. Dry toluene (1 mL) was used to complete the transfer. The resulting solution was stirred at 25° C. for 47 h.

### Example 7

### 5-(2-Chlorophenyl)-1,3-dihydro-7-fluoro-8-methoxy-2H-1,4-benzodiazepine-2-one

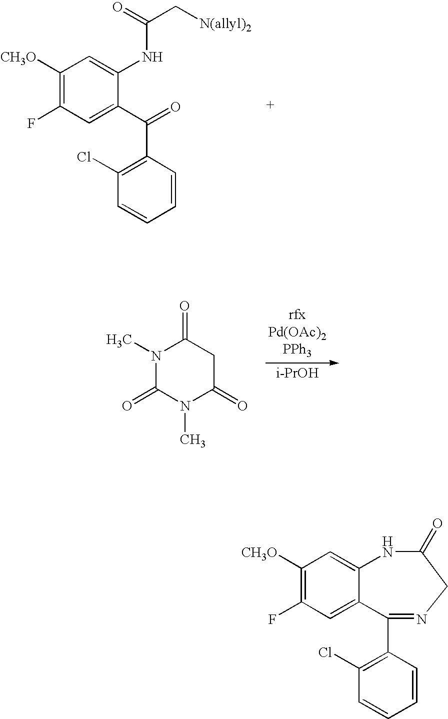

The crude diallylamine (20.00 g, 47.98 mmol), 10.49 g (67.17 mmol, 1.40 equiv) 1,3-dimethylbarbituric acid, 75.5 mg (0.288 mmol, 0.600 mol %) triphenylphosphine, 10.8 mg (0.0481 mmol, 0.100 mol %) palladium acetate and 63 mL isopropanol are charged to the flask. Acetic acid (0.51 mL, 0.54 g, 9.0 mmol, 0.20 equiv) is added and the suspension is then refluxed (bath 82° C., reaction mixture 75-76° C.) for 6.5 h. The suspension was then cooled to 25° C. and stirred overnight.

Toluene (125 mL) is added. The azeotrope is distilled (bath to 135° C.) (pot temperature to 109.1° C., head temperature to 99° C.) (128 mL collected/leaves ˜60-65 mL toluene in pot). The suspension is cooled to 25° C. and 50 ml H₂O containing 1.15 g (28.8 mmol) NaOH is added. The suspension is stirred for 60 min. The precipitate is suction filtered, washed with 15 mL of 25° C. H₂O twice and 15 mL of 25° C. toluene, and air dried 3 h to afford 14.40 g (94.2%) of beige-pale yellow solid.

Recrystallization from toluene to affords beige crystals, m.p. 210-212.5° C.; 500 MHz ¹H NMR (CDCl₃) δ 9.75 (br s, 1H), 7.51-7.49 (m, 1H), 7.41-7.34 (m, 3H), 6.76 (d, J=11.5 Hz, 1H), 6.71 (d, J=7.0 Hz, 1H), 4.38 (br s, 2H), 3.94 (s, 3H); 125 MHz ¹³C NMR (CDCl₃) δ 171.3, 169.6, 150.9 (d, J=12.4 Hz), 148.4 (d, J=246 Hz), 138.8, 135.5 (d, J=2.4 Hz), 133.5, 131.2, 131.1, 130.4, 127.2, 120.6 (d, J=5.6 Hz), 116.5 (d, J=19.5 Hz), 105.4 (d, J=1.9 Hz), 56.8 (d, J=28.1 Hz); IR (KBr) 3197, 3097, 3012, 2972, 2933, 2867, 1669, 1626, 1612, 1591, 1578, 1522, 1271 cm⁻¹. Elem. Anal. Calcd for C₁₆H₁₂ClFN₂O₂: C, 60.30; H, 3.79; Cl, 11.12; F, 5.96; N, 8.79. Found: C, 60.56; H, 3.80; N, 8.72.

### Example 8

### 5-(2-Chlorophenyl)-7-fluoro-2,8-dimethoxy-3H-1,4-benzodiazepine

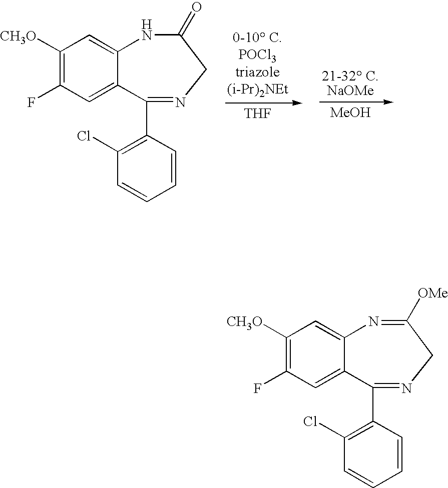

The flask is charged with 58.03 g (182.1 mmol) of the benzodiazepine (palladium content 23.34 ppm), 75.45 g (1.092 mol, 6.00 equiv) 1,2,4-triazole (T46108, [288-88-0], mw=69.07), 580 mL (516 g at 0.889 g.mL) dry THF, and 192 mL (143 g, 1.10 mol, 6.06 equiv) diisopropylethylamine. The yellow suspension is cooled to 5° C. (ice-water bath) and 25.0 mL (41.9 g, 273 mmol, 1.50 equiv) phosphorus oxychloride is added dropwise over 62 min at 5-8° C. The orange suspension is allowed to warm to 25° C. and stirred for 2 h.

Sodium methoxide (25 wt % in methanol) (292 mL, 275 g, 1.28 mol, 7.00 equiv) is added in one portion. The temperature rises from 24° C. to 36° C. and a thick yellow suspension forms. Methanol (160 mL) is added and the suspension stirred for 19 h.

Water (295 mL) is added. The temperature rises to 31.5° C. The organic solvent is then distilled at bath to 40° C. and 120-50 mm Hg (collected 1072 mL). The beige suspension is cooled to 0-5° C. (ice water bath) for 30 min. The precipitate is suction filtered, washed with 2×150 mL of 5° C. H₂O and 80 mL of 5° C. methanol, then air dried at 25° C. for 90 min and dried in a vacuum oven using house vacuum at 30° C. for ˜36 h to afford 59.51 g (98.2%) of off-white solid.

### Example 9

### 2-(Allylamino)-2′chloro-5-fluoro-4-methoxybenzophenone

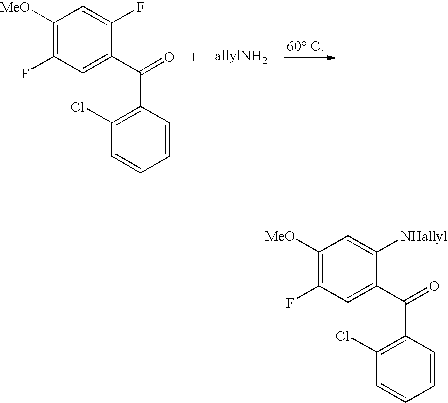

A mixture of 69.5 g (245.9 mmol) and 184 mL (140 g, 2.46 mol, 10.0 equiv) of allylamine was heated in a pressure bottle at 60° C. for 24 h. The suspension was allowed to cool then diluted with 500 mL H₂O and 200 mL heptane. The precipitate was suction filtered, washed with 80 mL H₂O then 80 mL heptane, and air dried 4 h at 25° C. to afford 73.83 g (93.9%) of yellow solid (CDCl₃).

Recrystallization from isopropanol (hot filter) (to 0° C.) affords bright yellow flakes, m.p. 125-127° C.; 500 MHz ¹H NMR (CDCl₃) δ 9.36 (br, 1H), 7.44 (dd, J=8.0 Hz, J=1.5 Hz, 1H), 7.38 (t, J=7.5 Hz, J=2.0 Hz, 1H), 7.34 (dd, J=7.0 Hz, J=1.5 Hz, 1H), 7.28 (dd, J=8.0 Hz, J=2.0 Hz, 1H), 6.85 (d, J=13.0 Hz, 1H), 6.18 (d, J=7.0 Hz, 1H), 6.02-5.95 (m, 1H), 5.37 (dm, J=17.0 Hz, J=2.0 Hz, 1H), 5.27 (dm, J=10.5 Hz, J=1.5 Hz, 1H), 3.97 (m, 2H), 3.91 (s, 3H); 125 MHz ¹³C NMR (CDCl₃) δ 194.8 (d, J=2.8 Hz), 155.0 (d, J=12.5 Hz), 151.4, 143.0 (d, J=234 Hz), 139.9, 134.2, 130.8, 130.5, 130.1, 128.5, 127.0, 120.4 (d, J=19.0 Hz), 116.9, 109.1 (d, J=4.8 Hz), 95.0, 56.2, 45.8; IR (KBr) 3281, 2979, 2940, 2857, 1632, 1607, 1564, 1525, 1260 cm⁻¹. Elem. Anal. Calcd for C₁₇H₁₅ClFNO₂: C, 63.85; H, 4.73; Cl, 11.09; F, 5.94; N, 4.38. Found: C; H; N.

### Example 10

### 2-Amino-2′-chloro-5-fluoro-4-methoxybenzophenone

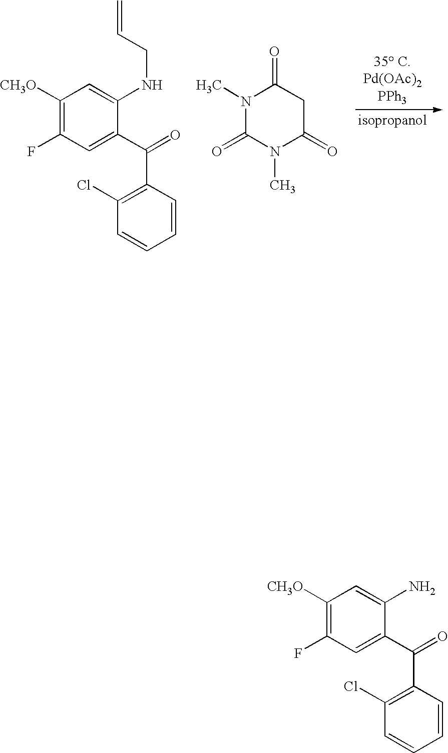

A 100 mL three-neck round bottom flask (septum with dry N₂ needle, septum with Teflon-coated thermocouple, septum, magnetic stirbar) is charged with 5.00 g (15.64 mmol) of the allylamine, 1.709 g (10.95 mmol, 0.7 equiv) 1,3-dimethylbarbituric acid, 7.0 mg (0.0312 mmol, 0.2 mol %) palladium acetate, 49.2 mg (0.188 mmol, 0.12 mol %) triphenylphosphine, and 50 mL isopropanol. The solution was heated at 35° C. for 1 h.

The mixture is cooled then concentrated on a rotary evaporator at 30° C. and 40 mm Hg. Toluene (50 mL) and 30 ml H₂O are added. Sodium hydroxide solution (30 wt %) is added dropwise to bring the pH to 12. The layers are separated. The organic layer is washed with 25 mL H₂O then concentrated in vacuo (rotary evaporator at 30° C. and 25 mm Hg then vacuum pump at 25° C. and 1 mm Hg for 3 h) to afford 5.89 g of yellow solid.

### Example 11

### 2-Bromo-2′-(2-chlorobenzoyl)-4′-fluoro-5′-methoxyacetanilide

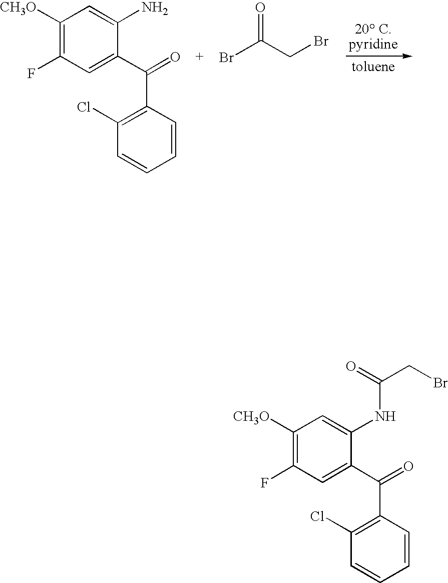

Bromoacetyl bromide (1.49 mL, 3.47 g, 17.2 mmol, 1.10 equiv) is added dropwise at 17-23° C. (ice H₂O bath) over 6 min to a suspension of 5.89 g of the crude amine and 1.39 mL (1.36 g, 17.2 mmol, 1.10 equiv) pyridine in 25 mL toluene. The resulting suspension is stirred at 17-23° C. for 2.5 h.

Water (20 mL) is added and the suspension stirred at 17-23° C. for 30 min. The suspension is cooled to 0-5° C. and stirred for 30 min. The precipitate is suction filtered, washed with 10 mL of 25° C. H₂O and 50 mL of 5° C. toluene, and air dried 2 h at 25° C. to afford 5.65 g (90.1% for two steps) of pale yellow solid.

### Example 12

### N,N-Diallylglycine Ethyl Ester

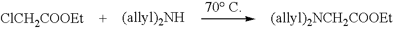

A mixture of 58 mL (66.7 g, 0.544 mol) ethyl chloroacetate and 201.5 mL (159.0 g, 1.64 mol, 3.00 equiv) diallylamine was stirred at 70° C. for 4.5 h.

The suspension was cooled to 35° C. and heptane (500 mL) was added. After cooling the resulting suspension to 25° C., the precipitate was suction filtered, washed with 100 mL heptane, and air dried to afford 70.68 g (97.2%) of beige flakes.

The combined mother liquors are concentrated on a rotary evaporator at 30° C. and 30-10 mm Hg then vacuum pump at 25° C. and 1 mm Hg for 15.5 h to afford 96.94 g (97.2%) of yellow oil.

### Example 13

### 2-(N,N-Diallylamino)-2′-(2-chlorobenzoyl)-4′-fluoro-5′-methoxyacetanilide

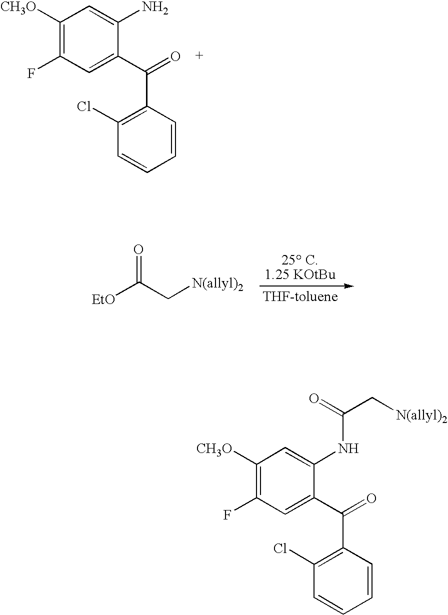

Potassium tert-butoxide in THF (27.0 mL, 25.3 g of 20 wt %, 5.05 g, 45.0 mmol, 1.25 equiv) was added dropwise via syringe at 20-25° C. (cool water bath) over 12 min to a suspension of 10.00 g (35.8 mmol) of the aminobenzophenone and 7.22 g (39.4 mmol, 1.10 equiv) crude ethyl N,N-diallylglycinate in 20 ml dry toluene. The resulting solution was stirred at 20-25° C. for 35 min.

The mixture was quenched by addition of 20 mL of saturated aqueous ammonium chloride. Water (30 mL) and toluene (30 ml) were added and the layers separated. The organic layer was washed with 30 mL H₂O then concentrated in vacuo (rotary evaporator at 30-40° C. and 70-25 mm Hg) to afford 17.58 g of orange oil.

Isopropanol (100 ml) was added and azeotrope was distilled at atmospheric pressure (under dry N₂) (bath 100° C.) (49 mL collected). The solution was cooled and seeded, stirred at 25° C. for 30 min, then stirred at 0° C. for 30 min. The precipitate was suction filtered, washed with 15 ml of 0° C. isopropanol, and air dried at 25° C. for 3 h to afford 13.21 g (crude) of very pale yellow solid (88.7%).

### Example 14

### 5-(2-Chlorophenyl)-1,3-dihydro-7-fluoro-8-methoxy-2H-1,4-benzodiazepine-2-one

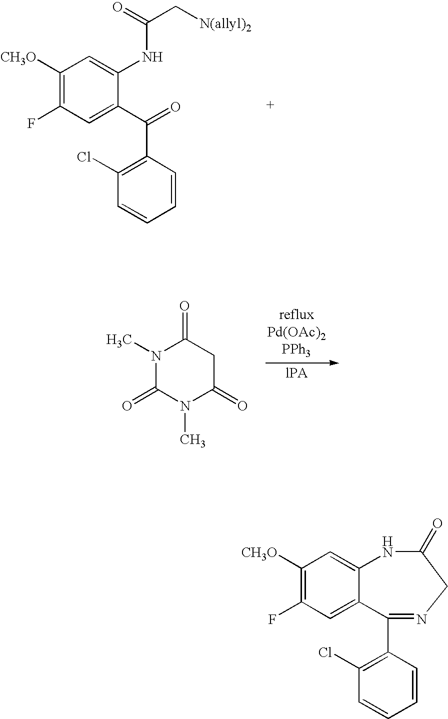

The crude diallylamine (35.8 mmol) in 48 mL isopropanol, 8.38 g (53.7 mmol, 1.50 equiv) 1,3-dimethylbarbituric acid, 56.3 mg (0.215 mmol, 0.600 mol %) triphenylphosphine, 8.1 mg (0.036 mmol, 0.100 mol %) palladium acetate, and 5 mL isopropanol are charged to the flask. Acetic acid (0.41 mL, 0.43 g, 7.2 mmol, 0.20 equiv) is added and the suspension is refluxed for 7 h (bath 82° C., pot 71° C.). The suspension is cooled to 25° C. and stirred overnight.

Toluene (95 mL) is added. The azeotrope is distilled (bath to 130° C.) (pot temperature to 107.0° C., head temperature to 97° C.) (91 mL collected/leaves ˜57 mL toluene in pot). The solution is cooled to 25° C. (a precipitate forms) and 38 ml H₂O containing 1.00 g (25 mmol) NaOH is added. The suspension is stirred for 60 min. The precipitate is suction filtered, washed with 12 mL of 25° C. H₂O twice and 12 mL of 25° C. toluene, and air dried 4 h at 25° C. to afford 9.80 g of crude beige solid (85.9%).

### Example 15

### N,N-Diallylaminoacetamide

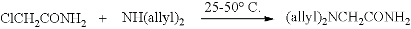

A suspension of 20.00 g (214 mmol) of 2-chloroacetamide and 79 ml (62.3 g, 642 mmol, 3.0 equiv) diallylamine was heated at 50° C. for 2.5 h.

Toluene (250 mL) and 50 mL H₂O containing 8.56 g (214 mmol) NaOH were added at 35° C. to produce two layers The layers were separated. The toluene layer was washed with 50 mL H₂O three times then concentrated in vacuo (rotary evaporator at 30° C. and 25 mmHg then vacuum pump at 25° C. and 1 mmHg for 17 h) to afford 29.71 g of pale yellow needles. This solid was recrystallized from toluene (total volume 100 mL) (to 0° C.) to afford 27.99 g of long colorless needles after 26 h vacuum dry at 25° C. and 1 mmHg (94.2%).

### Example 16

### 2,5-Difluoro-4-methoxybenzophenone

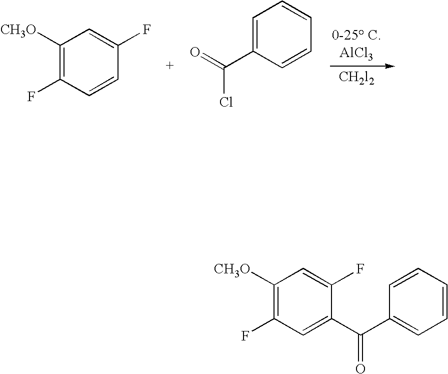

Aluminum chloride (23.07 g, 173 mmol) is charged to the reaction flask. Dichloromethane (150 mL) is added and the suspension cooled to 2° C. (ice-H₂O bath). Benzoyl chloride (20.1 mL, 24.4 g, 173 mmol) is added dropwise at 0-5° C. over 17 min. The suspension is stirred 10 min at 0-5° C. The difluoroanisole (25.0 g, 173 mmol) is then added dropwise at 0-5° C. over 15 min. The resulting solution is stirred at 0-5° C. for 2 h then allowed to warm to 25° C. and stir for 17.5 h.

The solution is poured over 200 g ice in a 500 mL flask. Dichloromethane (25 mL) is used to complete the transfer. The layers are separated and the aqueous layer is extracted with 25 mL dichloromethane. The combined organic layers are backwashed with 50 mL H₂O then dried (MgSO₄), filtered, and concentrated in vacuo (rotary evaporator at 25° C. and 150 mm Hg then vacuum pump at 25° C. and 1 mm Hg for 21.5 h) to afford 38.90 g of pale yellow crude solid (90.6%). The solid is recrystallized from 500 ml hexanes (with stirring to 25° C.) (to 0° C.) to afford 33.93 g of bright colorless flakes after 3 h dry at 1 mmHg and 25° C.

An analytical sample is prepared by recrystallization from hexanes twice more (to 25° C.) to afford large colorless needles, m.p. 76-77° C.; 500 MHz ¹H NMR (CDCl₃) δ 7.80 (m, 2H), 7.60 (tm, J=7.5 Hz, 1H), 7.47 (tm, J=7.5 Hz, 2H), 7.37 (dd, J=6.0 Hz, J=11.0 Hz, 1H), 6.74 (dd, J=6.5 Hz, J=11.0 Hz, 1H) 3.95 (s, 3H); 125 MHz ¹³C NMR (CDCl₃) δ 191.9, 157.4 (d, J=252 Hz), 152.0, 148.6 (dd, J=244 Hz, J=2.9 Hz), 138.0, 133.4, 129.80, 129.79, 128.6, 117.8 (J=21.0 Hz, J=4.8 Hz), 101.8 (dd, J=28.1 Hz, J=1.9 Hz), 56.9; IR (KBr) 3020, 2979, 2941, 1662, 1630, 1596, 1579, 1506 cm⁻¹.

### Example 17

### 2-(Benzylamino)-5-fluoro-4-methoxybenzophenone

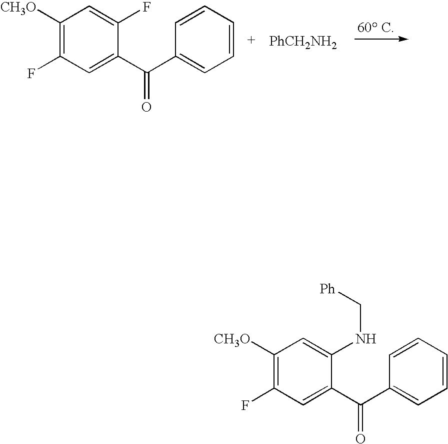

A mixture of 31.65 g (127.5 mmol) of the difluorobenzophenone and 55.7 mL (54.7 g, 510 mmol, 4.00 equiv) of benzylamine was heated at 60° C. for 48 h. The mixture was heated at 80° C. for 5 days. The suspension was cooled to 25° C., diluted with 170 mL heptane and 170 mL H₂O, and stirred for 60 min. The precipitate was suction filtered (150 mL coarse sintered glass funnel). 30 ml of 12 N HCl was added to the liquors to reach aqueous pH=1, 00 mL toluene added to dissolve the oil in the organic layer, and the layers separated. The organic layer was washed with 100 ml H₂O twice then concentrated on a rotary evaporator at 30° C. and 25 mm Hg to afford 40-45 g of orange syrup. Hexanes (100 mL) and some seed crystals were added. After stirring at 25° C. for 30 min, the precipitate was suction filtered to afford 30-35 g of slightly sticky yellow solid. Hexanes (100 mL) was added and the suspension stirred at 25° C. for 30 min. The precipitate was suction filtered to afford ˜27 g of less sticky yellow solid. This solid was recrystallized from isopropanol (175 mL) (hot filter) (to 25° C.) to afford yellow crystals.

The solid was further recrystallized from hexanes-heptane (hot filter) (to 0° C.) to afford 18.38 g (43.0%) of yellow crystals.

This solid (2.00 g) was further recrystallized from (50 mL) heptane (50 mL) (to 25° C.) to afford 1.913 g of yellow crystals after drying in vacuo (4.5 h at 25° C. and 1 mm Hg), mp. 109-5-112.8° C.; (¹H NMR) (CDCl₃) δ 9.45 (br t, 1H), 7.57-7.27 (m, 10H), 7.23 (d, J=13.0 Hz, 1H), 6.15 (d, J=7.5 Hz, 1H), 4.50 (d, J=6.0 Hz, 1H), 3.79 (s, 3H); (¹³C NMR) (CDCl₃) δ 197.3 (d, J=2.4 Hz), 154.1 (d, J=11.9 Hz), 151.0, 142.7 (d, J=233 Hz), 140.7, 138.5, 130.8, 129.1, 128.8, 128.4, 127.7, 127.4, 121.1 (d, J=19.1 Hz), 109.4 (d), 95.6, 56.1, 47.7; IR (KBr) 3450, 3286, 3059, 3013, 2967, 2929, 2860, 1630, 1608, 1595, 1562, 1523, 1248 cm⁻¹. Elem. Anal. Calcd for C₂₁H₁₈FNO₂: C, 75.21; H, 5.41; N, 4.18. Found: C, 75.52; H, 5.58; N, 4.19.

### Example 18

### 3-Acetyl-5-(2-chlorophenyl)-7-fluoro-2,8-dimethoxy-3H-1,4-benzodiazepine

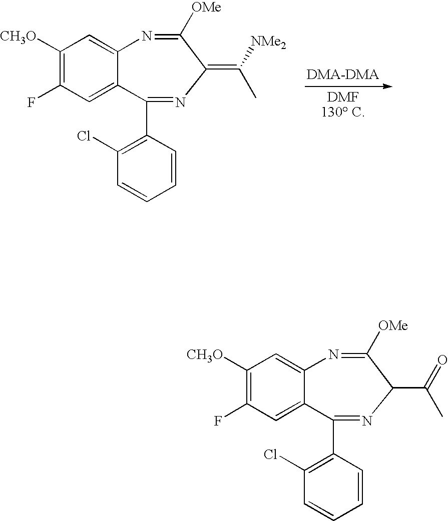

A solution of the imidate 26.0 g (78.1 mmol) in dry DMF (150 ml) is heated to 130° C. N,N-Dimethylacetamide dimethyl acetal (25.4 mL, 23.14 g of 90 wt %, 156 mmol, 2.00 equiv) (DMA-DMA) is added slowly over 22-23 h using a syringe pump. The dark solution is then heated at 130° C. (bath) for 9 h.

The dark solution is then cooled to 10° C. and the ice-water bath is removed. Toluene (300 mL) and water (250 mL) are added. The suspension is stirred 30 min at 25° C. The layers are separated. Water (150 mL) is added to the organic layer and the suspension stirred 30 min at 25° C. The layers are separated. Toluene is distilled from the organic layer at 30° C. (bath) and 25 mm Hg (collected 270 ml) to afford 45.46 g of red oil. Methanol (300 mL) is added and methanol is distilled at 30° C. (bath) and 85 mm Hg (collected 313 mL) to afford 40.98 g of red oil.

Methanol (100 mL) and water (4.22 mL, 4.22 g, 234 mmol, 3.0 equiv) are added and the solution stirred at 25° C. for 3 h. The precipitate is suction filtered, washed with 25 mL methanol, and air dried 1 h at 25° C. to afford 22.49 g (76.8%) of orange-tan solid.

Recrystallization from methanol (hot filter) (to 25° C.) affords pale yellow needles, mp. 172.5-174° C.; 500 MHz ¹H NMR (CDCl₃) δ 7.50-7.48 (br d, 1H), 7.40-7.34 (m, 3H), 6.87 (d, J=7.5 Hz, 1H), 6.77 (d, J=12.0 Hz, 1H), 4.1-3.9 (v br, 1H), 3.96 (s, 3H), 3.89 (s, 3H), 2.58 (br, 3H); 125 MHz ¹³C NMR (CDCl₃) δ 167.9, 157.2, 150.6 (d, J=12.0 Hz), 148.0 (d, J=245 Hz), 144.3, 138.4, 133.9, 131.8, 131.1, 130.4, 127.1, 121.1 (d, J=5.8 Hz), 115.6 (d, J=19.0 Hz), 110.3 (d, J=1.9 Hz), 70.9, 56.4, 55.6, 28.9.

### Example 19

### 3-Acetyl-5-(2-chlorophenyl)-7-fluoro-2,8-dimethoxy-3H-1,4-benzodiazepine Semicarbazone

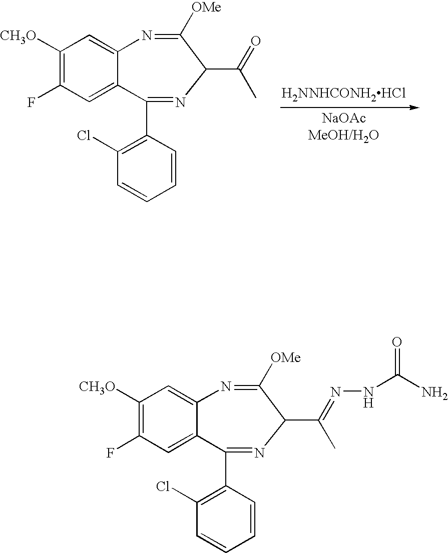

A solution of 1.488 g (13.34 mmol, 1.00 equiv) of semicarbazide hydrochloride in 4.0 mL H₂O is added over 6 min to a suspension of 5.000 (13.34 mmol) of the ketone and 1.121 g (13.34 mmol) sodium bicarbonate in 20 mL methanol. The resulting thick near-colorless suspension is stirred at 25° C. for 3.5 h.

The precipitate is suction filtered, washed with 10 mL H₂O, washed with 10 mL methanol, and air dried 10 h at 25° C. to afford 5.60 g (97.2%) of colorless solid.

Recrystallization from methanol (hot filter) (to 0° C.) affords colorless crystals, mp>180° C. gradually turns yellow then >190° C. melts to a red liquid with gas evolution; 500 MHz ¹H NMR (CDCl₃) δ 8.26 (br, 1H, NH), 7.49 (br d, 1H), 7.40-7.33 (m, 3H), 6.90 (d, J=8.0 Hz, 1H), 6.78 (d, J=12.0 Hz, 1H), 4.30 (br, 1H), 3.97 (s, 3H), 3.88 (s, 3H), 2.24 (s, 3H); 125 MHz ¹³C NMR (CDCl₃) δ 167.0, 158.8, 157.4, 150.2 (d, J=12.4 Hz), 148.4, 147.7 (d, J=244.6 Hz), 143.9 (d, J=2.5 Hz), 138.4, 133.6, 131.6, 130.7, 130.1, 126.8, 121.3 (d, J=5.8 Hz), 115.3 (d, J=19.1 Hz), 110.1 (d, J=1.5 Hz), 67.7, 56.2, 55.1, 14.0.

### Example 20

### 3-Acetyl-5-(2-chlorophenyl)-7-fluoro-2,8-dimethoxy-3H-1,4-benzodiazepine Semicarbazone 29

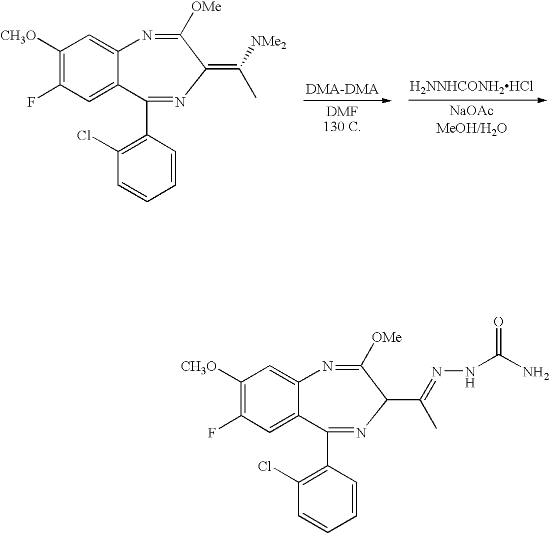

A solution of the imidate 26.0 g (78.1 mmol) in dry DMF (150 ml) is heated to 130° C. N,N-Dimethylacetamide dimethyl acetal (25.4 mL, 23.14 g of 90 wt %, 156 mmol, 2.00 equiv) (DMA-DMA) is added slowly over 23-24 h using the syringe pump. The dark solution is then heated at 130° C. (bath) for 16.5 h.

The dark solution is cooled to 10° C. and the ice-water bath is removed. Toluene (300 mL) and water (250 mL) are added. The suspension is stirred 30 min at 25° C. The layers are separated. Water (150 mL) is added to the organic layer and the suspension stirred 30 min at 25° C. The layers are separated. Toluene is distilled from the organic layer at 30° C. (bath) and 25 mm Hg (collected 274 ml) to afford 41.94 g of red oil. Methanol (300 mL) is added and methanol is distilled at 30° C. (bath) and 85 mm Hg (collected 313 mL) to afford 39.57 g of red oil.

Methanol (100 mL) is added to the red oil and the solution transferred to the 250 mL flask. Methanol (20 mL) is used to complete the transfer. Water (4.22 mL, 4.22 g, 234 mmol, 3.00 equiv) is added and the solution stirred at 25° C. for 3.5 h. A solution of 8.710 g (78.1 mmol) of semicarbazide hydrochloride in 19.5 mL H₂O is prepared then added over 5-10 min at 25° C. (cool H₂O bath). The mixture is stirred at 25° C. for 3.5 h. The precipitate is suction filtered, washed with 65 mL H₂O, washed with 65 mL methanol twice, then air dried 11 h at 25° C. to afford 28.62 g (84.8%) of beige solid.

### Example 21

### Carbamoyl-5-(2-chlorophenyl)-7-fluoro-1,2-dihydro-8-methoxy-3-methylpyrazolo[3,4-b][1,4]benzodiazepine 30

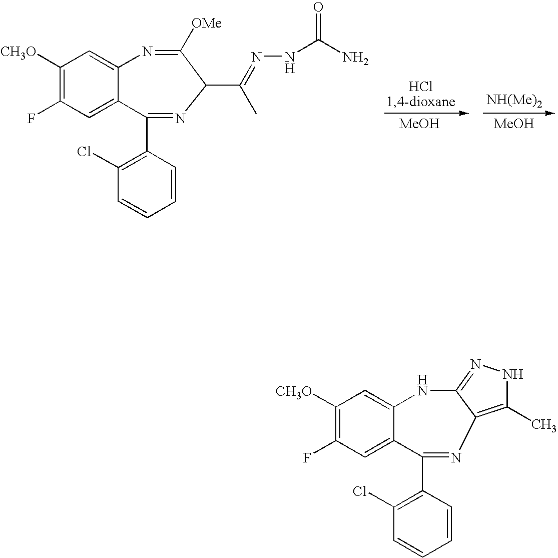

Hydrogen chloride in 1,4-dioxane (0.19 mL of 4.0 M, 0.76 mmol, 33 mol %) is added to a suspension of 1.000 g (2.32 mmol) of the crude MAI2 Semicarbazone in 10 mL dry methanol. The suspension is stirred at 25° C. for 4.5 h (dry N₂). Dimethylamine in methanol (0.38 mL of 2.0 M, 0.76 mmol, 33 mol %) is added, and the resulting suspension stirred at 25° C. for 30 min. The suspension is cooled to 0-5° C. and stirred for 30 min. The precipitate is suction filtered, washed with 2.0 mL of 0-5° C. methanol, and air dried 1.5 h at 25° C. to afford 750 mg (81.0%) of bright orange solid. Recrystallization from methanol (hot filter) (to 25° C.) affords red crystals.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

What is claimed:

1. A method of making a compound of Formula I 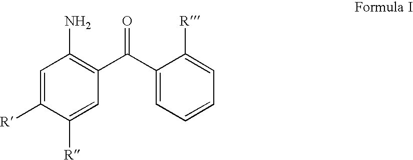
comprising the steps of:
(a) reacting a compound of formula II 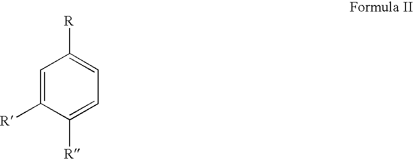
with a compound of Formula III 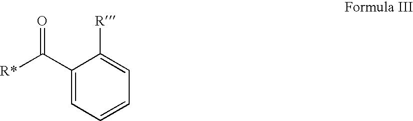
to form a benzophenone of Formula IV 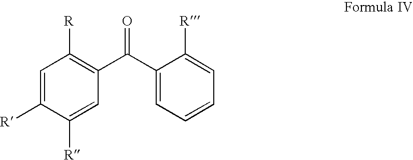
(b) reacting the benzophenone of Formula IV with a benzyl or allyl amine to form a compound of formula V; and 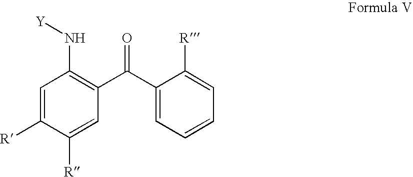
(c) reacting the compound of Formula V with acid to cause N-displacement of Y to form the compound of Formula I; 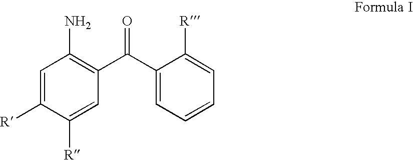
wherein:
Y is benzyl or allyl;
R is halogen, tosylate, or mesylate;
R* is halogen;
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 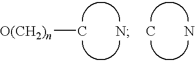
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

2. A method of forming a compound of Formula I comprising the step of 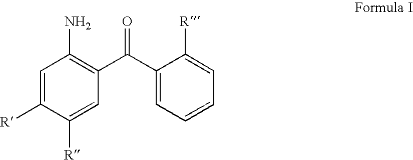
reacting a compound of Formula V 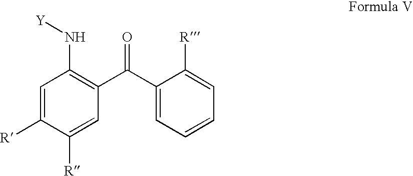
with acid under conditions sufficient to cause hydrogenolysis to form the compound of Formula I;
wherein:
Y is benzyl or allyl;
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 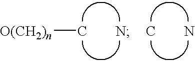
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

3. The method of claim 2, wherein the acid is sulfuric acid.

4. The method of claim 3, wherein the sulfuric acid is 98% sulfuric acid.

5. The method of claim 2, wherein the acid is hydrobromic acid.

6. The method of claim 5, wherein the conditions sufficient to cause hydrogenolysis comprise the addition of a palladium catalyst.

7. The method of claim 6, wherein the palladium catalyst is Pd(OH)₂ on carbon.

8. The method of claim 7, wherein the Pd(OH)₂ on carbon is activated with H₂.

9. A compound of Formula VI 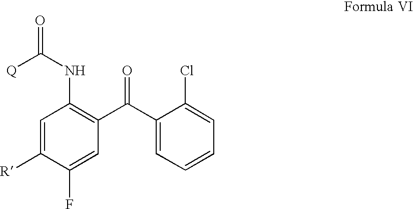
wherein:
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 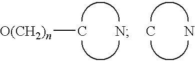
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
Q is (Q′)₂ N(Q″)ᵣ-;
Q′ is lower alkyl, lower alkenyl, lower alkoxy, aryl, aryl alkyl, heteroaryl, heterocyclyl, or cycloalkyl, optionally substituted with one or more substituents selected from the group consisting of halogen, alkyl, OH, and alkoxy;
Q″ is a lower alkylenyl linking group; and
r is 1, 2, or 3.

10. The compound of claim 9, wherein R′ is —OMe.

11. The compound of claim 10, wherein Q″ is —CH₂— and r is 1.

12. The compound of claim 11, wherein Q′ is allyl.

13. The method of claim 1, further comprising the step of reacting a compound of Formula I 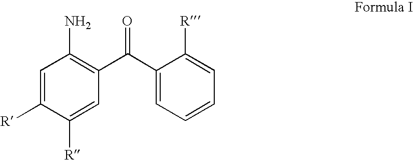
with Q-C(O)O-Q′″ under mild reaction conditions in the presence of a base to form a compound of Formula VI; 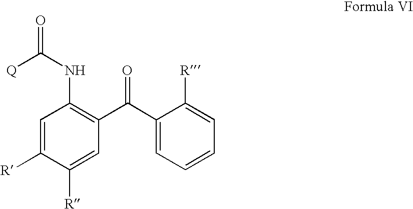
wherein:
Q is (Q′)₂ N(Q″)ᵣ-;
Q′ is lower alkyl, lower alkenyl, lower alkoxy, aryl, aryl alkyl, heteroaryl, heterocyclyl, or cycloalkyl, optionally substituted with one or more substituents selected from the group consisting of halogen, alkyl, hydroxyl, and alkoxy;
Q″ is a lower alkylenyl linking group;
r is 1,2, or 3;
Q′″ is lower alkyl;
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 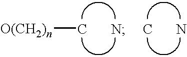
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

14. The method of claim 13, wherein Q″ is —CH₂— and r is 1.

15. The method of claim 13, wherein Q′ is allyl.

16. The method of claim 13, wherein Q′″ is ethyl.

17. A method of making a compound of Formula VI, 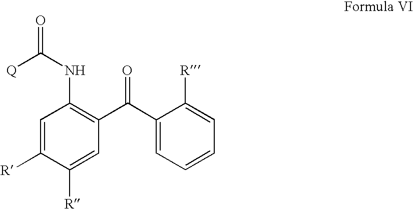
comprising the step of reacting a compound of Formula I 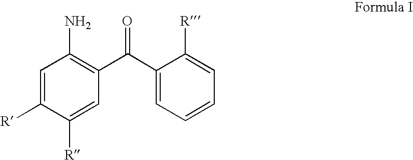
with Q-C(O)O-Q′″ under mild reaction conditions in the presence of a base;
wherein:
R″ is alkyl, alkoxy, halogen, COOH, COAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 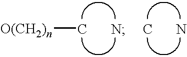
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring;
R′″ is hydrogen, halogen, CN, NO₂, alkyl, or alkoxy;
Q is (Q′)₂ N(Q″)ᵣ-;
each Q′ is independently H, lower alkyl, lower alkenyl, lower alkoxy, aryl, aryl alkyl, heteroaryl, heterocyclyl, or cycloalkyl, optionally substituted with one or more substituents selected from the group consisting of halogen, alkyl, hydroxyl, and alkoxy;
Q″ is a lower alkylenyl linking group;
r is 1, 2, or 3; and
Q′″ is lower alkyl.

18. The method of claim 17, wherein the mild reaction conditions include a temperature of between 20° and 30° C.

19. The method of claim 17, wherein Q″ is —CH₂— and r is 1.

20. The method of claim 17, wherein Q′ is allyl.

21. The method of claim 17, wherein Q′″ is ethyl.

22. The method of claim 17, wherein the base is a metal alkoxide.

23. The method of claim 22, wherein the metal alkoxide is potassium tert-butoxide.

24. The method of claim 15, further comprising the step of deallylation-cyclization of the compound of Formula VI 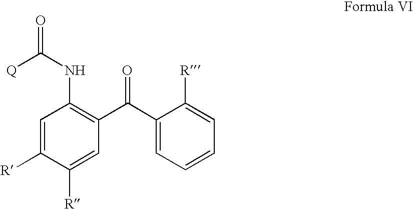
to form a compound of Formula VII

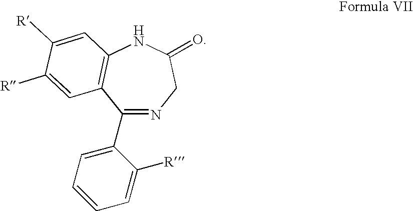

25. A method of making a compound of Formula VII, 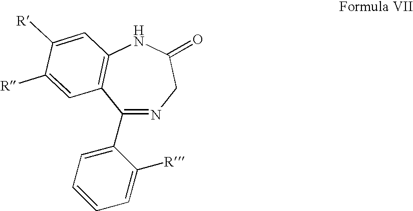
comprising the step of deallylation-cyclization of a compound of Formula VI 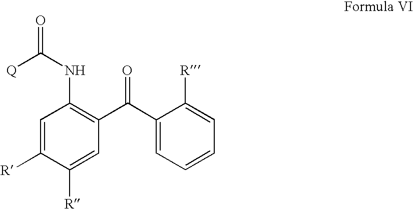
wherein:
Q is (Q′)₂ N(Q″)ᵣ-;
Q″ is —CH₂—;
Q′ is allyl;
r is 1;
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 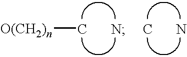
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

26. A method of making a pyrazolo[3,4-b][1,4]benzodiazepine comprising the step of converting a compound of Formula VII 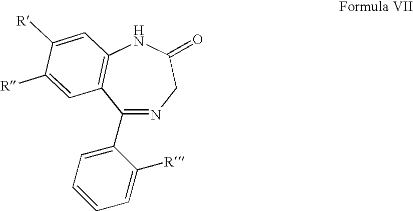
to an imidate of Formula VIIa; 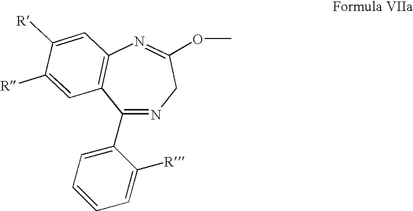
wherein
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 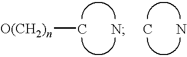
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

27. The method of claim 26, further comprising the step of reacting the imidate of Formula VIIa with dimethylacetamide dimethylacetal to form an enamine of Formula VIIb

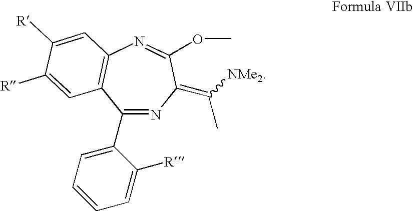

28. The method of claim 27, further comprising the step of reacting the enamine of Formula VIIb with semicarbazide hydrochloride to form the semicarbazone of Formula VIIc

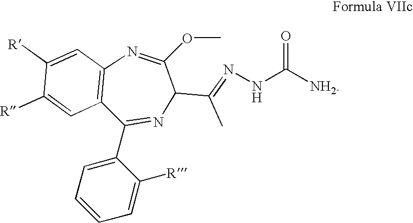

29. The method of claim 28, further comprising the step of converting the semicarbazone of Formula VIIc to a carbamate of Formula VIId by acid-catalyzed cyclization

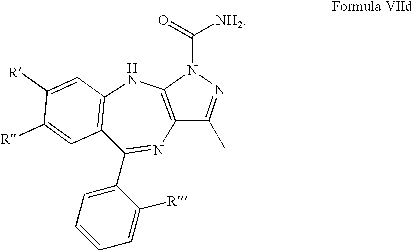

30. The method of claim 29, further comprising the step of converting the carbamate of formula VIId to the pyrazolo[3,4-b][1,4]benzodiazepine of Formula VIII by acyl transfer to an amine

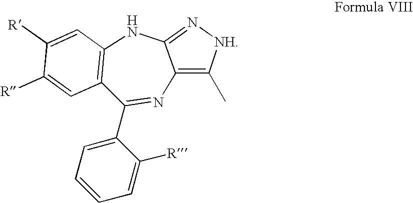

31. A method of making a semicarbazone of Formula VIIc comprising the step of reacting an enamine of Formula VIIb 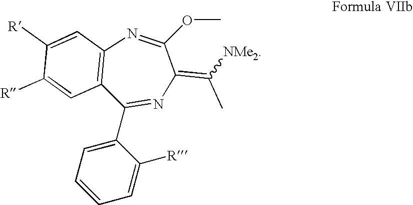
with semicarbazide hydrochloride to form the semicarbazone of Formula VIIc; 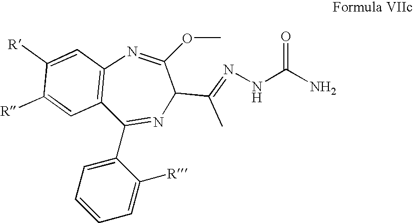
wherein
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 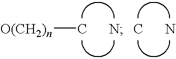
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

32. The method of claim 31, wherein
R′ is methoxy;
R″ is F; and
R′″ is Cl.

33. A method of making a carbamate of Formula VIId 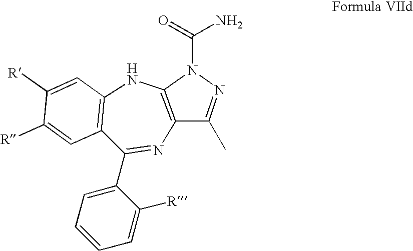
comprising the step of converting a semicarbazone of Formula VIIc 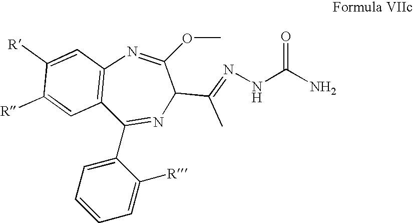
to the carbamate of Formula VIId by acid-catalyzed cyclization;
wherein
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 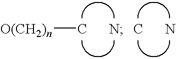
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

34. The method of claim 33, wherein
R′ is methoxy;
R″ is F; and
R′″ is Cl.

35. The method of claim 33, wherein the acid-catalyzed cyclization is HCl-catalyzed cyclization.

36. A method of making a pyrazolo[3,4-b][1,4]benzodiazepine of Formula VIII 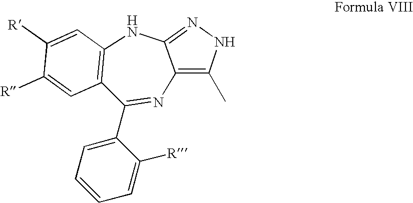
comprising the step of converting a carbamate of formula VIId 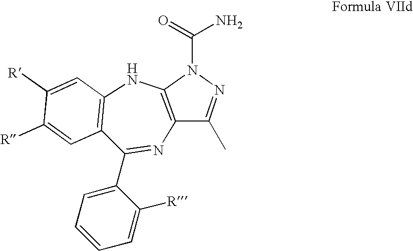
to the pyrazolo[3,4-b][1,4]benzodiazepine of Formula VIII by acyl transfer to an amine.

37. The method of claim 36, wherein
R′ is methoxy;
R″ is F; and
R′″ is Cl.

38. The method of claim 27, further comprising the step of converting the enamine of Formula VIIb to a ketone of Formula VIIe 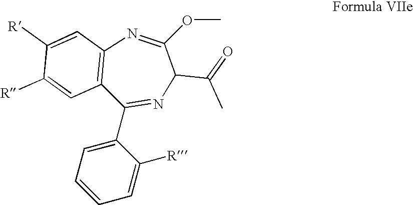
by hydrolysis in methanol.

39. The method of claim 38, further comprising the step of reacting the ketone of Formula VIIe with semicarbazide hydrochloride to form a semicarbazone of Formula VIIc

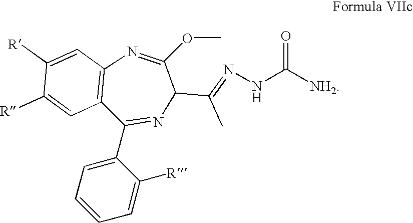

40. A compound of the formula VIIe 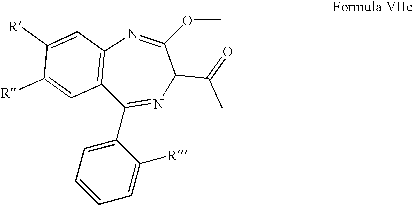
wherein:
R″ is alkyl, alkoxy, halogen, COOH, COAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 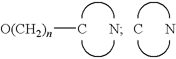
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

41. The compound of claim 40, wherein
R′ is methoxy;
R″ is F; and
R′″ is Cl.

42. A compound of the formula VIIc 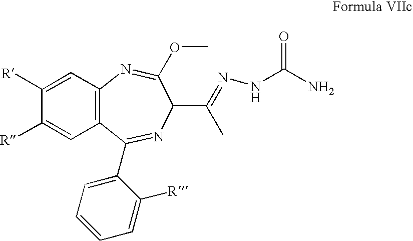
wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 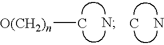
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

43. The compound of claim 42, wherein
R′ is methoxy;
R″ is F; and
R′″ is Cl.

44. A compound of the formula VIId 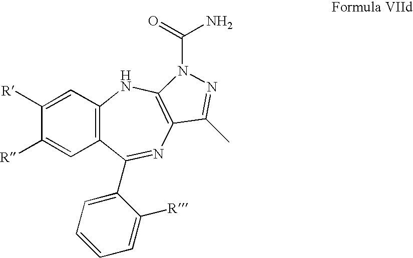
wherein:
R″ is alkyl, alkoxy, halogen, COOH, COAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 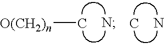
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

45. The compound of claim 44, wherein
R′ is methoxy;
R″ is F; and
R′″ is Cl.

46. A method of making a compound of Formula VIIe, 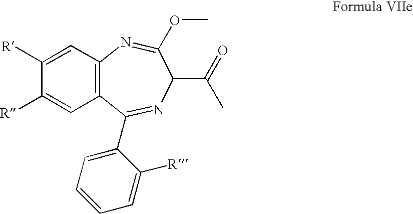
comprising deriving a compound of Formula VIIe from an enamine of Formula VIIb 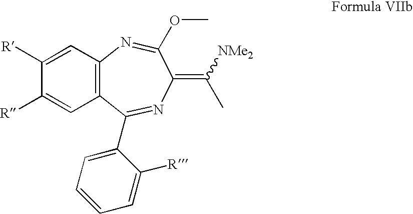
wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 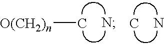
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

47. The method of claim 46, wherein
R′ is methoxy;
R″ is F; and
R′″ is Cl.

48. A method of making a carbamate of Formula VIId, 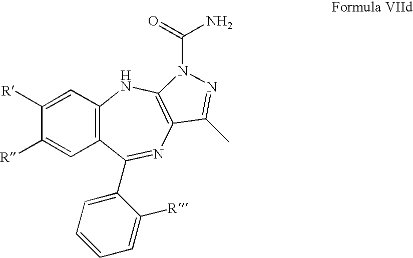
comprising converting a semicarbazone of Formula VIIc 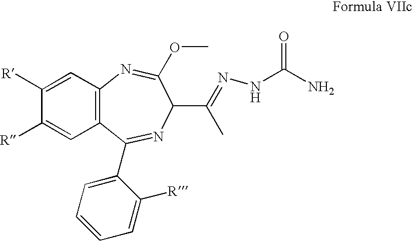
to the carbamate of Formula VIId by acid-catalyzed cyclization
wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 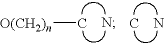
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

49. The method of claim 48, further comprising the step of converting the carbamate of formula VIId 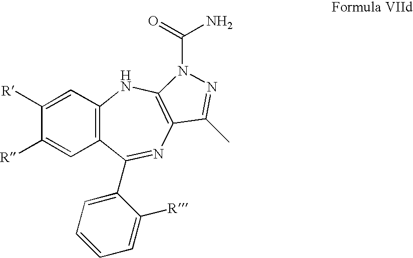
to a pyrazolo[3,4-b][1,4]benzodiazepine of Formula VIII 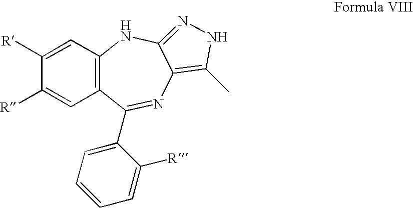
by acyl transfer to an amine.

50. The method of claim 49, wherein
R′ is methoxy;
R″ is F; and
R′″ is Cl.

51. A method of making a pyrazolo[3,4-b][1,4]benzodiazepine of Formula VIII, 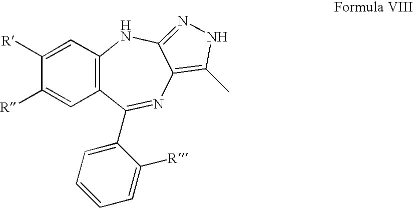
comprising the step of converting a carbamate of formula VIId 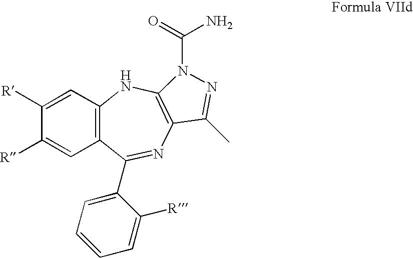
to the pyrazolo[3,4-b][1,4]benzodiazepine of Formula VIII by acyl transfer to an amine wherein:
R″ is alkyl, alkoxy, halogen, COOH, COOAlkyl, CN, C(O)N(R⁶)₂, or (OCH₂CH₂)ₙOCH₃;
R′ is alkyl, halogen, alkyl substituted by halogen, OH, alkoxy, alkoxy substituted by halogen, phenyl, N(R⁶)₂, (OCH₂CH₂)ₙOCH₃, O(CH₂)ₘNR⁷R⁸, or 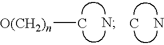
is a 6-membered heterocycle optionally substituted with alkyl or C(O)OR⁶;
each R⁶ is independently hydrogen or alkyl;
each n is independently 1, 2, or 3;
m is 2, 3, or 4;
R⁷ and R⁸ are each independently hydrogen, alkyl, or alkoxyalkyl;
or R⁷ and R⁸ taken together with the nitrogen to which they are attached form a 6-membered heterocycle which is optionally substituted by alkyl;
or R′ and R″ together form a 5-membered heterocyclic ring; and
R′″ is hydrogen, halogen, CN, NO₂, alkyl, or alkoxy.

52. The method of claim 51, wherein
R′ is methoxy;
R″ is F; and
R′″ is Cl.

53. The method of claim 51, wherein the amine is dimethylamine.

54. The method of claim 53, wherein
R′ is methoxy;
R″ is F; and
R′″ is Cl.

55. A method of N-debenzylation comprising reacting an N-benzylamine with HBr in the presence of a catalyst.

56. The method of claim 55, wherein the catalyst is a palladium catalyst.

57. The method of claim 55, wherein the palladium catalyst is Pd(OH)₂ on carbon.
